(19) United States
(12) Patent Application Publication
(10) Pub. No.: US 2005/0009103 A1
(43) Pub. Date: Jan. 13, 2005

(54) Methods of precise molecular-level determination of ligand-receptor interactions and designing selective drug compounds based on said interactions

(75) Inventors: Dennis A. Dougherty, South Pasadena, CA (US); Henry A. Lester, South Pasadena, CA (US); Nima Shiva, Los Angeles, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Neurion Pharmaceuticals, Pasadena, CA (US)

(21) Appl. No.: 10/886,742

(22) Filed: Jul. 8, 2004

Related U.S. Application Data
(60) Provisional application No. 60/485,773, filed on Jul. 8, 2003.

(51) Int. Cl. G01N 33/53; A61K 38/16; C07K 14/705

(52) U.S. Cl. 435/7.1; 514/12; 530/324

(57) ABSTRACT

The present invention discloses methods of determining highly precise interactions between a membrane protein receptor and various compounds. The methods of the present invention utilize a receptophore model system and nonsense codon suppression methods combined with heterologous in vivo expression in Xenopus oocytes.

### PRIORITY TO PROVISIONAL APPLICATION UNDER 35 U.S.C. §119(e)

This application claims priority under 35 U.S.C. §119(e) of provisional application Ser. No. 60/485,773 filed Jul. 8, 2003.

### STATEMENT AS TO RIGHTS TO INVENTIONS MADE UNDER FEDERALLY SPONSORED RESEARCH OR DEVELOPMENT

This invention was made with government support under NIH Grant No. NS34407, awarded by the Department of Health and Human Services. The government has certain rights in this invention.

### FIELD OF THE INVENTION.

The present invention generally relates to methods of obtaining high-precision structural and functional information on membrane bound receptors such as ligand-gated ion channel (LGIC) receptors and G-protein-coupled receptors (GPCRs). The present invention more specifically relates to methods for identifying the structural determinants associated with binding to these receptors as well as the associated physiological effects (e.g. agonist, partial agonist, antagonist, etc).

### BACKGROUND OF THE INVENTION

Membrane bound receptors are the targets for more than 60% of current drugs. These receptors include GPCR's, LGICs and other types of ion-channels. There are numerous lines of experimental evidence that indicate the existence of various modes by which ligands can modulate receptors. Modulatory ligand activity can be described, for example, as agonist, partial agonist, full agonist, inverse agonist, antagonist, allosteric agonist, allosteric modulator, or allosteric enhancer.

This diversity in ligand activity can be attributed to the number of different ways ligands can selectively bind to receptor subtypes. For example, molecules can interact at the endogenous ligand binding site or at a distinct site often referred to as an allosteric site. Traditionally, the more common approach in drug discovery has been to target the endogenous ligand binding site. However, drugs can also interact at a distinct site, which in some circumstances may offer advantages with regards to selectivity.

Furthermore, it has been shown that ligands can selectively stabilize or destabilize different receptor conformations. Selective affinity of a ligand for a receptor binding site in a specific receptor conformation state determines the physiological response of the host system to that ligand. Relating the structural basis of this selective affinity to a specific physiological response could lead to another dimension in control of the quality of ligand efficacy.

Studies with LGICs suggest allosteric modulators have several therapeutic advantages over ligand based inhibitors including the ability to modulate receptor activity through conformational changes in the receptor protein that are transmitted from the allosteric site to ligand binding site and/or directly to effector coupling sites.

The essential role of LGICs and GPCRs to neuronal signaling makes them ideal therapeutic drug targets. Specifically, drugs have been developed to interact with receptors in specific ways to modify receptor function and modulate synaptic transmission. However, there is still a need in the art to develop high-precision methods in which different ligand-induced receptor conformations can be identified and used to optimize drug-binding interactions. More specifically, there are realms where specific targeting of a particular state of a specific receptor subtype could be useful therapeutically.

Therefore, methods of determining modulatory ligand activity along with the corresponding molecular-level ligand-receptor interactions are highly desirable to the design of pharmaceutical drugs. The nature of integral membrane bound protein receptors makes it difficult to study structure-function relationships with traditional high-resolution structural methods such as x-ray crystallography or NMR spectroscopy. As a result, additional methods and approaches to identifying modulatory compounds is desired.

### BRIEF SUMMARY OF THE INVENTION

The methods described herein relate to the construction of a receptophore model using unnatural amino acid substitutions as an alternative, and further describe the use of the receptophore model to identify and refine potential ligands. One aspect of the invention incorporates unnatural amino acids in the native receptor to form an altered receptor and compares the effect of a selected compound upon the altered receptor.

The methods provided herein include the incorporation of unnatural amino acids with in vivo nonsense codon suppression. Methods of in vivo nonsense codon suppression are used to probe structure-function relationships in receptor binding sites. This invention uses the nonsense suppression methodology to modify receptors of interest and then evaluate or screen diverse molecules. Application of these methods to ligand gated ion channel receptors, including but not limited to, nicotinic acetylcholine receptors and serotonin 5-HT3 receptors, and to G-protein-coupled receptors, will elucidate and control several important characteristics of ligand binding to receptors.

The present invention will provide not only the ability to determine the details of how, where, and in what conformation a ligand binds to its receptor, it will also provide the ability to correlate a specific receptor conformation with a desired physiological response of the host system to that ligand. Based on this information, the present invention also will allow for the design, through high-precision compound modifications, of state-selective agonists that stabilize a preferred ligand-receptor conformation, thereby leading to the identification and continued development of improved drug classes. Methods of precise molecular-level determination of the modulatory ligand activity, along with the corresponding molecular level ligand-receptor interactions, are disclosed. More specifically, methods of determining the specific physiological effect of a compound on the activity of its receptor using a nonsense suppression methodology are disclosed herein. Furthermore, methods of incorporating unnatural amino acids into binding and regulatory sites of the receptor expressed in intact cells are provided, so that structure-function relationships between the receptor and its ligand may be probed.

An aspect of the invention is to provide a method of determining the specific physiological effect of a compound on the activity of its receptor comprising developing a receptophore model, wherein said model allows for generation of compounds that can selectively modulate a receptor subtype in a specific receptor conformation to achieve a desired physiological activity; using nonsense suppression methodology to determine details of the nature and location of receptor binding of said compounds; and using said receptophore model to predict which compound could achieve said physiological activity on the target receptor by evaluating how, where and in what receptor conformation state said compound binds to the receptor.

Another aspect of the invention to provide a method of determining the nature of a compound's interaction with a receptor comprising: a) incorporating unnatural amino acids into binding and regulatory sites of the receptor, resulting in an altered receptor; b) measuring the compound's ability to bind to the altered receptor; and c) comparing the results of step (b) to the same compound's ability to bind to an unaltered receptor.

The invention also provides a method of altering a compound so that it interacts with its receptor to achieve desired ligand activity comprising: a) determining the nature of the compound's interaction with the receptor; b) analyzing how and where the compound interacts with the receptor; and c) based on the analysis in step (b), chemically modifying the compound to achieve desired ligand activity.

A further aspect of the invention provides a screening methodology comprising a GPCR or LGIC membrane protein receptor which has been modified to replace native amino acids with unnatural amino acids, wherein the receptor is expressed in vivo by Xenopus oocytes.

### BRIEF DESCRIPTION OF THE DRAWINGS

FIG. 1 provides a scheme showing incorporation of unnatural amino acids into the receptor in a Xenopus oocyte expression system.

FIG. 2 provides a plot of log[EC₅₀/EC_{50(WT)}] vs. cation-π binding ability at α-Trp149 of the nicotinic acetylcholine receptor for the wild type trp and the fluorinated trp derivatives 5-F-Trp, 5,7-F2-Trp, 5,6,7-F₃-Trp and 4,5,6,7-F₄-Trp.

FIG. 3 provides various derivations of tyrosine.

FIG. 4 provides various derivations of phenylalanine.

FIG. 5 provides various derivations of methionine and threonine.

FIG. 6 provides various derivations of glutamic acid.

FIG. 7 provides the distances in angstroms (Å) between specified atoms.

FIG. 8 provides a plot of log[EC₅₀/EC₅₀(WT)] versus calculated cation-π ability is plotted for the series of fluorinated Trp derivatives at Trp α149.

FIG. 9a-c provides a scheme showing the hydrogen bond analysis of nAChR.

FIG. 9a provides a scheme showing the backbone amide carbonyl of Thr α150 (X═NH) is replaced with an ester carbonyl Tah α150 (X═O).

FIG. 9b provides a scheme showing representative voltage-clamp current traces for oocytes expressing nAChRs suppresses with Thr or Tah at α150.

FIG. 9c provides a scheme showing representative epibatidine dose-response relations and fits to the hill equation for nAChR suppressed with Thr (∘) and Tah(•).

FIG. 10 provides a scheme showing the crystal structure data (X-Ray) and computational modeling (Calculated) of agonist binding. The (a) labeled positions show the calculated distance for a cation-π interaction. The (b) labeled positions show the calculated distance for an N⁺—H or N⁺C—H hydrogen bond with the backbone carbonyl. The (c) labeled positions show the calculated distance for a C_{aromatic}—H•••O═C hydrogen bond with the backbone carbonyl.

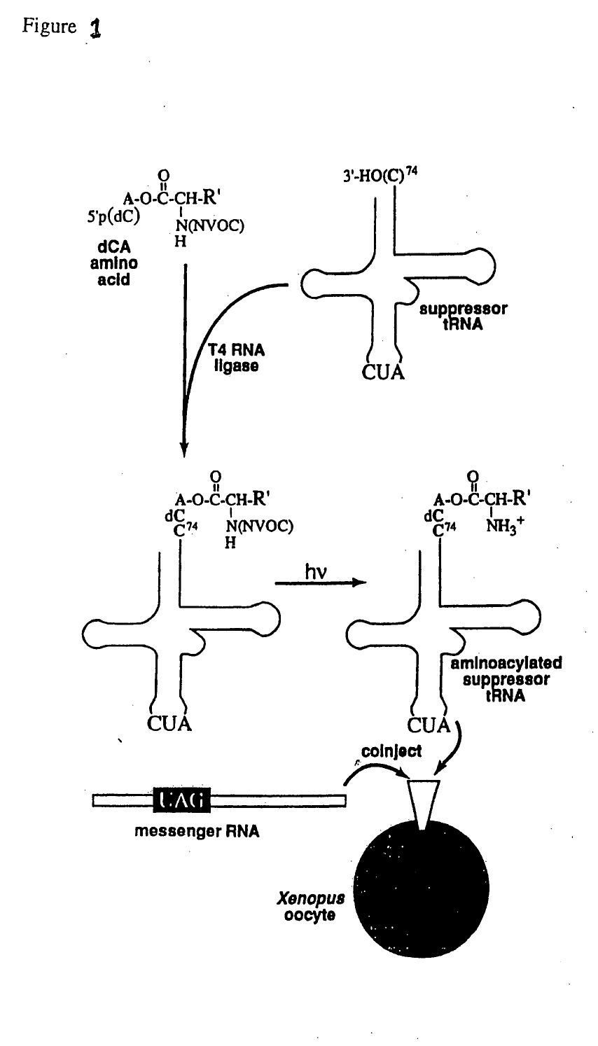

### DETAILED DESCRIPTION OF THE INVENTION

The present invention describes a method of obtaining precise binding and interaction information of ligands or drugs with their receptors by developing receptophore models to identify the specific physiological effect of a compound on the activity of its receptor. The information elucidated from these novel experiments will allow the generation of compounds that can selectively modulate a receptor subtype in a specific receptor conformation to achieve a desired ligand activity.

Ligand-gated ion channel (LGIC) receptors and G-protein-coupled receptors (GPCRS) are integral membrane proteins of the central and peripheral nervous systems that function as receptors for small neurotransmitter molecules. Cell to cell communication in the brain occurs at a synapse, which is a small gap between two neurons. A neurotransmitter is released from the presynaptic neuron, diffuses within the synaptic cleft, and then binds to receptors, such as LGICs and GPCRs, on the surface of the postsynaptic neuron.

Receptors within each LGIC or GPCR family share common structural features. LGICs are generally oligomeric and contain an integral ion channel. Examples of LGICs include nicotinic acetylcholine receptors, γ-aminobutyric acid (GABA) and glycine receptors, and 5-hydroxytryptamine (5-HT3) receptors. On the other hand, GPCRs commonly consist of one polypeptide composed of seven hydrophobic transmembrane domains connected by extracellular and intracellular loops. Although GPCRs may dimerize, dimerization may or may not affect their function. Examples of GPCRs include β-adrenoceptors, opioid receptors, dopamine receptors, and olfactory receptors (Wheatley, M. (1998) Essays Biochem. 33:15).

Ion channels are transmembrane proteins that regulate entry of various ions into cells from the extracellular matrix. Ion channels are physiologically important, playing essential roles in regulating intracellular levels of various ions and in generating action potentials in nerve and muscle cells. Hill, B. Ionic Channels of Excitable Membranes (Sinauer, Sunderland, Mass., 1992). Passage of ions through ion channels is characterized by selective filtering and by a gating-type mechanism which produces a rapid increase in permeability (Angelides, K. J. and Nuttov, T. J. (1981) J. Biol. Chem. 258:11858-11867). Ion channels may be wither voltage-gated, implying that current is gated (or regulated) by membrane potential (voltage), or chemically-gated (e.g., acetylcholine receptors and γ-aminobutyric acid receptors), implying that current is gated primarily by binding of a chemical rather than by the membrane potential (Butterworth, J. F. and Strichartz, G. R. (1990) Anesthesiology 72:711-734).

In addition to affecting action potentials, ion channels facilitate other important physiological functions such as cardiac pacemaking, neuron bursting, and possibly learning and memory (Crow, T. (1988)Trends Neurosci. 11:136-142: Hodgkin, A. I., and Huxley, A. F. (1952) J. Physiol. 117:500-544). In addition to their involvement in normal cellular homeostasis, ion channels are associated with a variety of disease states and immune responses. Diseases associated with dysfunction of ion channels include neurological disorders, metabolic diseases, cardiac diseases, tumor-driven diseases, and autoimmune diseases. Neurodegenerative disorders include epilepsy, stroke, cerebral ischemia, cerebral palsy, hypoglycemia, Alzheimer's disease, Huntington's disease, asphyxia and anoxia, as well as for the treatment of neuropathic pain, spinal cord trauma, and traumatic brain injury.

A ligand's activity depends in part on its specificity for particular receptor subtypes and the dynamics of ligand/receptor interactions. Based on their activity, ligands can be classified as full agonist, partial agonist, antagonist, inverse agonist, allosteric agonist, allosteric modulator or allosteric enhancer. A full agonist produces full receptor activation and the maximal system response. A partial agonist produces submaximal receptor activation, submaximal system response, and potential inhibition of full agonist activation. An antagonist produces no physiological response but blocks agonist activation. An inverse agonist is an antagonist in systems that are not constitutively active, but has the added property of reducing constitutive activity of a receptor that is not dependent on agonist activation. An allosteric agonist activates the receptor through a site that is distinct from that of the endogenous agonist. An allosteric modulator blocks the system response but does not necessarily interfere with the endogenous ligand-receptor interactions. Also, it does not mask the normal physiological effects, and since these sites are less conserved, provides greater opportunities for developing receptor subtype selective compounds. An allosteric enhancer potentiates the effects of agonists on the receptor, since it works in the presence of endogenous agents.

Ligands also can be selective for a specific receptor conformation. For example, Gether et al. found that receptor conformational changes altered fluorescence of ligands covalently labeled with an environment-sensitive fluoropore, indicating the compounds' different preferences for receptor conformation (Gether et al. (1995) J. Biol. Chem. 270:28268). In another example, Seifert et al. tested a series of β₂-adrenoceptor agonists for their ability to promote steps of the G-protein activation/deactivation cycle. The investigators attributed the difference between full agonist and partial agonist to the ability to stabilize distinct conformational states of the GPCR (Seifert et al. (2001) J. Pharmacol. Exp. Ther. 297(3):1218). These studies suggest that compounds selective for a particular receptor conformation may have therapeutic value.

As used herein, a “receptor” is a ligand-gated ion channel (LGIC) receptor or G-protein-coupled receptor (GPCR) or other membrane bound receptor protein.

As used herein, a “ligand” refers to a compound or drug that binds to a receptor. A ligand can be an endogenous neurotransmitter molecule, such as, for example, acetylcholine, dopamine, or serotonin. It also can be a therapeutic drug compound designed to have receptor binding properties. The efficacy of ligand binding is defined herein as the propensity to interact with a receptor in a specific conformation, leading to a specific physiological response.

As used herein an “endogenous binding site” is an endogenous ligand binding site on a membrane bound protein receptor.

As used herein an “allosteric site” is a modulatory binding site on a receptor that is topographically distinct from the endogenous ligand site.

As used herein, an “allosteric interaction” is a ligand/receptor interaction at an allosteric site that modulates the ligand binding at an endogenous ligand site.

As used herein, an “allosteric modulator” is a compound that affects either receptor function system response, or ligand/receptor interactions at an endogenous ligand site.

As used herein, a “receptophore” is the ensemble of steric and electronic features of a biological target that is necessary to ensure optimal supramolecular interactions with a specific ligand and to trigger or block the biological function of the target.

As used herein, an “unnatural amino acid” is not one of the 20 recognized natural amino acids as provided in Creighton, Proteins, (W.H. Freeman and Co. 1984) pp.2-53.

As used herein, an “nicotinic acetylcholine receptor” (nAChR) is a prototypical member of the Cys-loop family of LGIC, which also includes γ-aminobutyric acid, glycine and serotonin receptors.

As used herein, an embryonic muscle nAChR is a cylindrical transmembrane protein composed of five subunits (α1)₂, β1, γ and δ.

As used herein, an “acetylcholine binding protein” (AChBP) is a soluble protein homologous to the agonist binding site of the nAChR.

Generation of Receptophore Model

Integral membrane protein receptors contain many transmembrane segments. For this and other reasons, it is difficult to generate enough pure, properly folded, and functional proteins for high-resolution structural methods such as x-ray crystallography or NMR spectroscopy. The methods herein describe the construction of a receptophore model using unnatural amino acid substitutions as an alternative, and further describe the use of the receptophore model to identify and refine potential ligands.

An accurate receptophore model is built through identification of amino acids involved in the ligand binding site and the probing of the molecular forces involved. First, unnatural amino acids are incorporated into target receptors using nonsense suppression methodology. Altered receptors are expressed heterologously on Xenopus oocyte membranes or synthesized using in vitro translation mixtures. Compounds found to modulate a receptor subtype in a specific receptor conformation are screened for binding efficacy to the altered receptor. Electrophysiological or biochemical assays are used to measure the effects, if any, of unnatural amino acid substitutions on ligand binding. Binding data involving the wild-type versus the altered receptor are compared to define the molecular forces involved in receptor/ligand binding.

The interaction of acetylcholine with the nicotinic acetylcholine has been studied as described in Zhong et al. (1998) Proc. Natl. Acad. Sci. 95:12088-12093. An agonist receptophore model of the nicotinic receptor family can be developed after multiple agonist contact points are identified through systematic mapping of the target binding sites using the in vivo nonsense suppression method for unnatural amino acid incorporation. A number of aromatic amino acids have been identified as contributing to the agonist binding site, suggesting that cation-π interactions may be involved in binding the quaternary ammonium group of the agonist, acetylcholine. A compelling correlation has been shown between: (i) ab initio quantum mechanical predictions of cation-π binding abilities and (ii) EC50 values for acetylcholine at the receptor for a series of tryptophan derivatives that were incorporated into the receptor by using in vivo nonsense suppression method for unnatural amino acid incorporation. Such a correlation is seen at one, and only one, of the residues tested: tryptophan-149 of the α subunit. This finding indicates that, on binding, the cationic, quaternary ammonium group of acetylcholine makes van der Waals contact with the indole side chain of the α tryptophan- 149, providing the most precise structural information to date on this receptor.

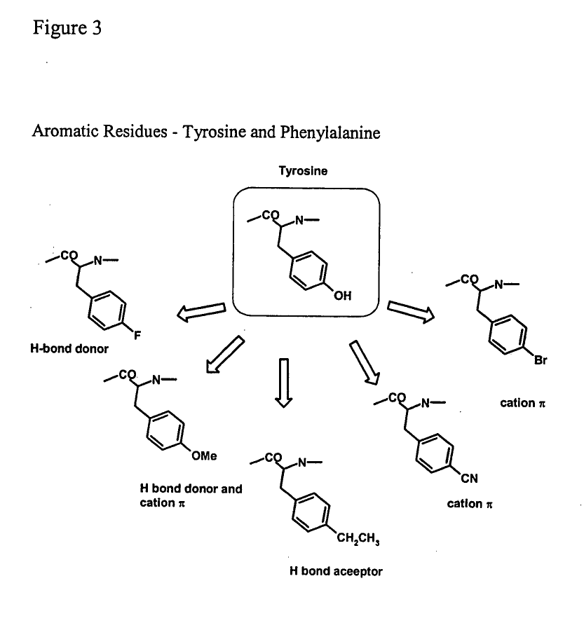

Unnatural amino acids are incorporated into the receptor binding sites through the use of nonsense codon suppression (Noren et al. (1989) Science 244:182; Nowak et al. (1998) Methods in Enzymol. 293:515), see FIG. 3. In the nonsense suppression method, two RNA species are prepared using standard techniques such as in vitro synthesis from linearized plasmids. The first is an mRNA encoding the receptor of interest but engineered to contain an amber stop codon (UAG) at the position where unnatural amino acid incorporation is desired. The second is a suppressor tRNA that contains the corresponding anticodon (CUA) and that is compatible with the expression system employed, such as Tetrahymena thermophila tRNAGln G73 for Xenopus oocytes or E. coli expression systems. The tRNA is then chemically acylated at the 3′ end with the desired unnatural amino acid using techniques known in the art such as that described in Kearney et al. (1996), Mol. Pharmacol., 50: 1401-1412. Unnatural amino acids also can be incorporated via site-directed mutagenesis.

Synthesis of the unnatural amino acids depends upon the desired structure. The unnatural amino acid may be prepared, for example, by modification of natural amino acids. Also, many unnatural amino acids are commercially available. A representative list of amino acids is as follows, and is not considered exhaustive: 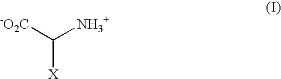
where X is selected from the group consisting of: 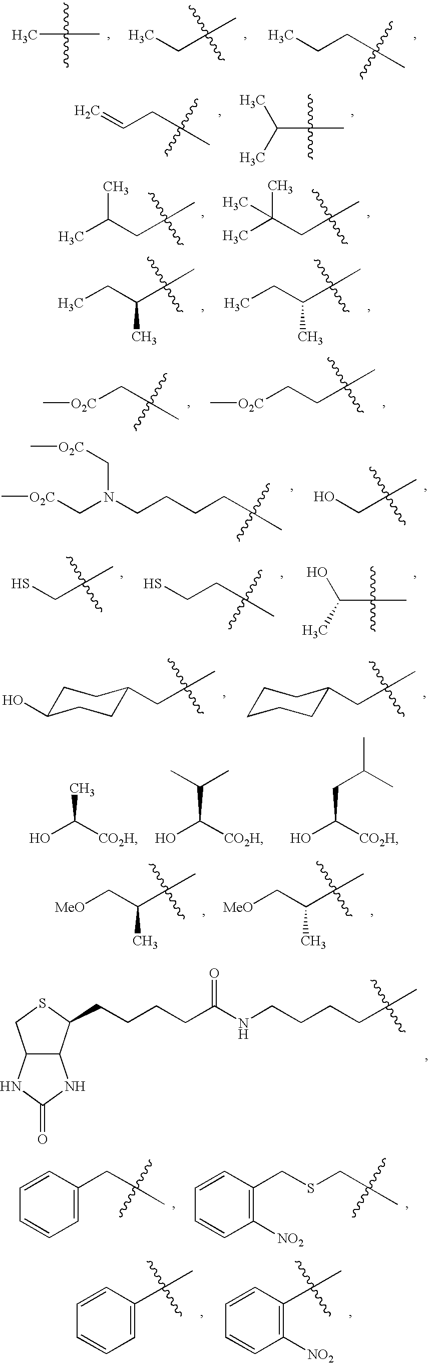 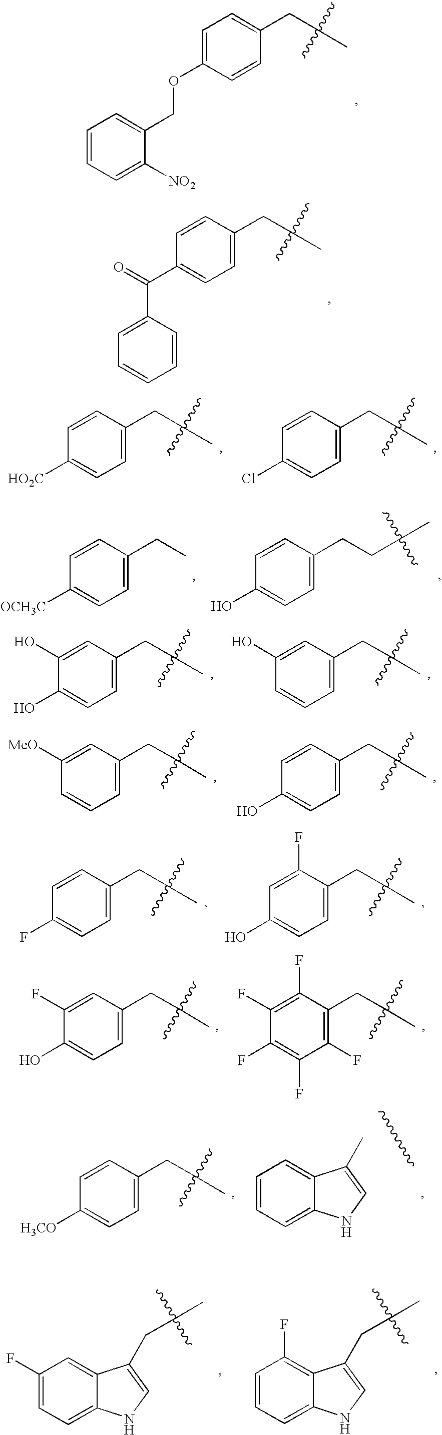 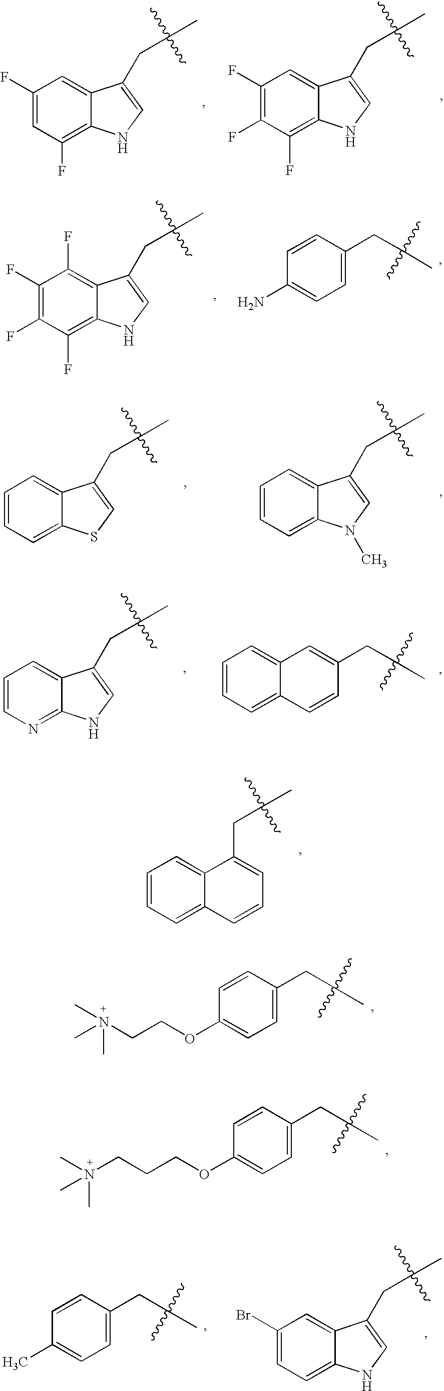 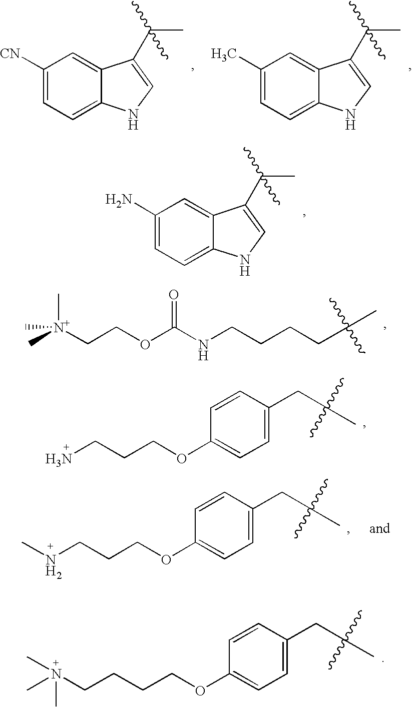
wherein: Y is CH2, (CH)n, N, O, or S, and n is 1 or 2. Examples of such compounds include, but are not limited to, the following compounds:

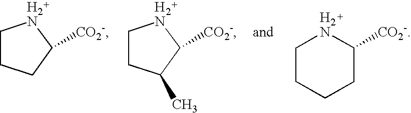

Note also that racemic amino acids can be used, because only L-amino acids, and not D-amino acids, are incorporated (Cornish, et al. (1995) Angew. Chem. Int. Ed. Engl. 34: 621-633).

In one embodiment, after synthesis of the relevant mRNA and acylated-tRNA, the species are co-injected into intact Xenopus oocytes such as those described in Nowak et al. (1998) Methods in Enzymol 293:515 using standard procedures known in the art. During translation the ribosome incorporates the unnatural amino acid into the nascent peptide at the position of the engineered stop codon, and an altered receptor is expressed on the oocyte membrane.

An electrophysiological method such as the voltage clamp is used to assess the ligand-binding capabilities of altered ion channel receptors. The voltage clamp assay measures ligand-binding to a receptor by detecting changes in the oocyte membrane potential that are induced by ion transport across the cell membrane. Such electrophysiological methods are well known in the art and have been used for the study of ion channels in the Xenopus oocyte expression system.

Other ligand-binding assays can be developed to measure ligand-receptor binding events that do not involve changes in membrane potential. The invention is not limited by the particular binding assay employed, since one skilled in the art can select a biochemical assay for use with a particular system, unnatural amino acids employed, receptor, ligand and target compound involved in a particular study.

For example, in one embodiment, a labeled ligand is used to physically detect the presence of the bound or unbound ligand. Various types of labels, including but not limited to, radioactive, fluorescent and enzymatic labels have been used in binding studies and are well known in the art. Labeled ligands can be commercially obtained or prepared using techniques known in the art. A binding assay using a radioactively labeled ligand may include the following steps: (1) incubating purified receptors or oocytes expressing receptors with the labeled ligand, (2) allowing an appropriate time for ligand-binding, (3) counting the number of bound ligands using a scintillation counter, and (4) comparing the differences in radioactive counts for altered and unaltered receptors.

Receptor/ligand binding data are compiled to create a model of a receptor/ligand binding event. The contribution of specific amino acid side chains to ligand binding is inferred from the comparative properties of a natural amino acid with the substituted unnatural amino acid. While one skilled in the art is capable of selecting an unnatural amino acid substitution to investigate a putative receptor/ligand interaction, here are some examples of how relevant information is extrapolated from these experiments.

(1) A cation-π interaction is important if fluoro-, cyano-, and bromo- amino acid derivatives, substituted for natural aromatic amino acids, abrogate ligand binding. When incorporated into an aromatic amino acid, these substituents withdraw electron density from the aromatic ring, weakening the putative electrostatic interaction between a positively charged group on the ligand and the aromatic moiety. Fluoro- derivatives are often preferred because fluorine is a strong electron-withdrawing group, and often adds negligible steric perturbations.

(2) Hydrophobic interactions at a given position are important if ligand binding is affected by substitutions that increase hydrophobicity without significantly altering the sterics of the side chain, thereby allowing the importance of hydrophobic interactions to be investigated in the absence of artificial steric constraints. One example of such a manipulation is conversion of a polar oxygen to a nonpolar CH₂ group, as in O-Methyl-threonine to isoleucine. Other methods to increase hydrophobicity, such as increasing side chain length, as in the substitution of allo-isoleucine for valine; or β-branch addition, as in the substitution of norvaline for isoleucine; or γ-branch addition, as in the substitution of t-butylalanine for isoleucine; may produce results that support the important of hydrophobic interactions.

(3) A local α-helix or β-sheet structure is important if an α-hydroxy acid substitution influences ligand binding. Incorporation of an α-hydroxy acid into the peptide backbone will produce an ester linkage instead of an amide bond. Since the amide hydrogen bond is important for stabilization of local α-helices and β-sheets, the α-hydroxy acid substitution disrupts these structures.

(4) By incorporating the phosphorylated or glycosylated analogue of a given amino acid into the receptor, the ligand binding in the presence or absence of the putative modification can be compared.

(5) Using photoreactive unnatural amino acids, the importance of specific side chains or protein modifications can be studied. For example, addition of the photoremovable nitrobenzyl group to the side chain of an amino acid can prevent interactions with the ligand, or may block side chain modifications such as phosphorylation and methylation. UV irradiation removes the nitrobenzyl group thereby restoring the amino acid to its native form. Therefore, ligand-binding measurements taken before and after UV irradiation can uncover side chain contributions to ligand binding. Similarly, the importance of local protein structures such as loops can be investigated by incorporating the unnatural amino acid (2-nitrophenyl) glycine (Npg). Irradiation of the Npg-modified amino acid triggers proteolysis of the protein receptor backbone. If UV irradiation disrupts ligand binding to the Npg-modified receptor, the unnatural amino acid indicates the importance of the related structure.

(6) Fluorescent reporter groups such as the nitrobenzoxadiazole (NBD) fluorophore or spin labels such as nitroxyl can be incorporated into the receptor using unnatural amino acids containing these labels. For example, after incorporation of an NBD-amino acid into the receptor, fluorescence resonance energy transfer between a fluorescently-labeled ligand and the NBD-amino acid can provide information such as the distance between the amino acid residue and the ligand-binding site.

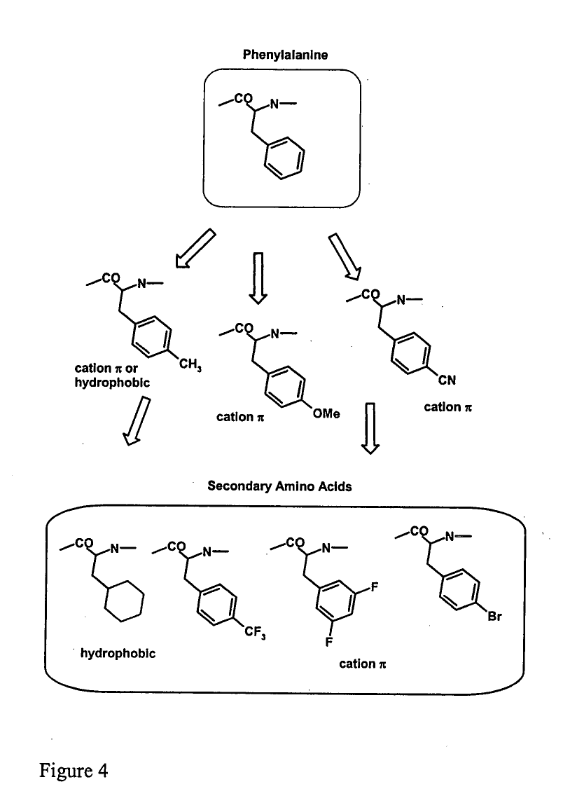

(7) For the tyrosine of phenylalanine position, the derivatives would include 4-OMe, 4-Br, and 4-CN, since the ability to participate in π-π, cation-π and hydrophobic interactions varies. See FIGS. 3-4.

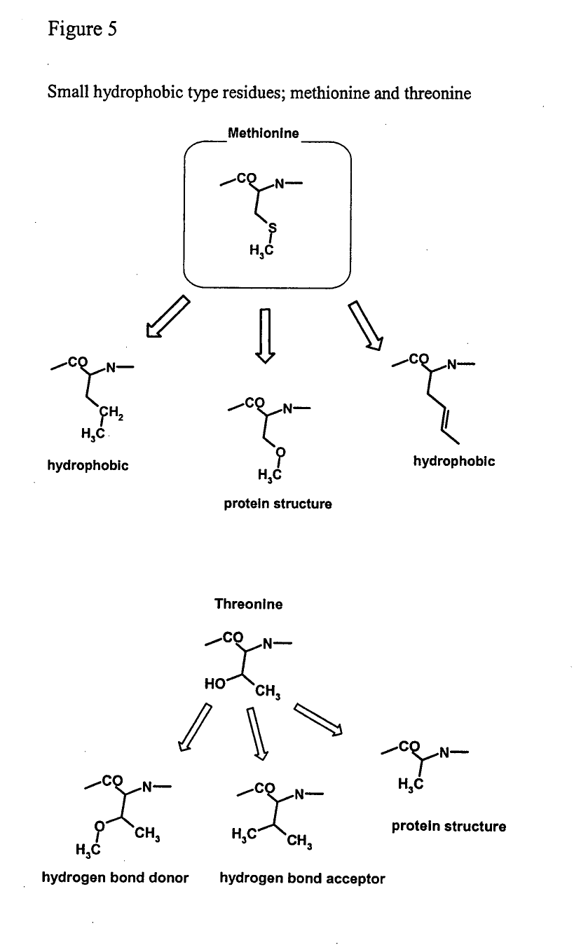

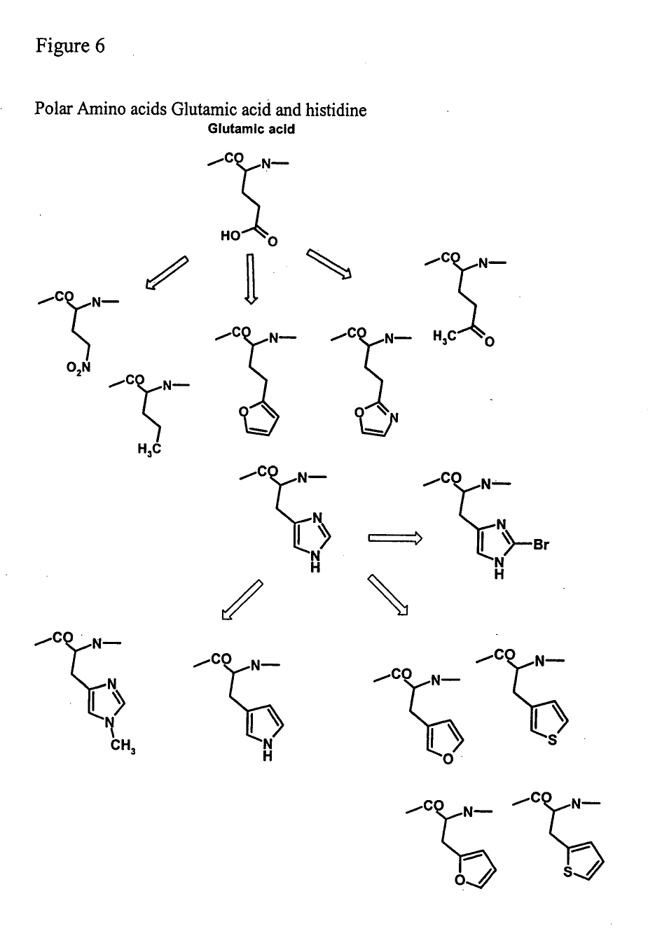

(8) Methionine, threonine, glycine, glutamic acid and histidine have the ability to interact with ligands in a manner different than the aromatic amino acids. More typically, the interactions are of a hydrophobic or hydrogen bond type. See FIGS. 5 and 6.

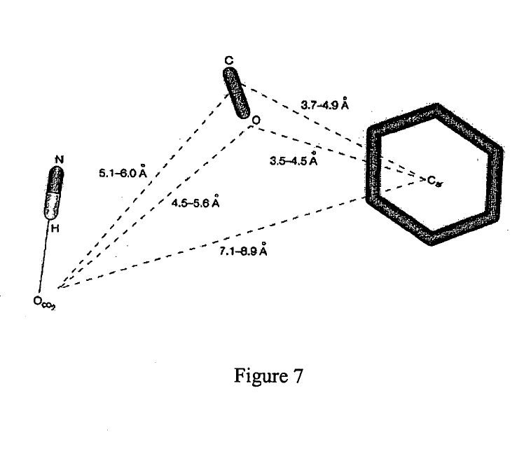

Preferably, the residues His, Gly, Glu, Tyr, Met and Phe are substituted with unnatural amino acids and the altered molecule or receptor evaluated by known allosteric modifiers or developed modifiers. See FIG. 7.

Identifying and Refining Compounds Specific for Receptor Subtypes and/or Conformations Using the Receptophore Model

The invention can be used to develop compounds that are specific for a receptor subtype and/or conformation. First, developing receptophore models for the interactions between receptor subtypes and a ligand that exhibits subtype specificity can identify amino acids that contribute to a ligand's subtype-selective binding. Second, developing receptophore models with receptors that assume different conformations and compounds that stabilize or are selective for a particular conformation can identify conformation-specific contacts. The data from these experiments can be used to engineer a more optimal compound by modifying the compound to take better advantage of subtype-specific or conformation-specific interactions. The following examples provide various modifications and their impact on the conformation or interaction.

(1) If the receptor/ligand model predicts stacking of an aromatic amino acid and an aromatic group of the ligand, a more parallel geometry between the aromatic groups may strengthen this interaction.

(2) If the receptor/ligand model suggests the importance of a specific hydrogen bond, a stronger hydrogen-bonding group can be substituted to increase the ligand's affinity for the receptor.

(3) If the ligand contains groups that sterically hinder its binding, these groups can be removed in favor of smaller or other non-sterically hindering groups.

(4) If hydrophobic forces contribute to the interaction at a particular position, less polar or larger hydrocarbon groups can be substituted within the steric limitations of the binding site.

(5) If an aromatic group in the binding site is left unengaged by the inhibitor, a positively charged group in the appropriate geometry for a cation-π interaction may increase the compound's affinity for the binding site.

The compounds can be evaluated by their effects on various aspects of the receptor, such as their effects on physiological activity. Physiological activity is measured by a change of agonist or ligand potency, efficacy or single channel kinetics.

The following examples are provided for illustration purposes, and are not intended to be limiting.

### EXAMPLE 1

Materials:

DNA oligonucleotides were synthesized on an Expedite DNA synthesizer (Perceptive Biosystems, Framingham, Mass.). Restriction endonucleases and T4 ligase were purchased from New England Biolabs (Beverly, Mass.). T4 polynucleotide kinase, T4 DNA ligase, and Rnase inhibitor were purchased from Boehringer Mannheim Biochemicals (Indianapolis, Ind.). I³⁵S-methionine and ¹⁴C-labeled protein molecular weight markers were purchased from Amersham (Arlington Heights, Ill.). Inorganic pyrophosphatase was purchased from Sigma (St. Louis, Mo.). Stains-all was purchased from Aldrich (Milwaukee, Wis.). T7 RNA polymerase was either purified using the method of Grodberg and Dunn (1988) J. Bact. 170:1245 from the overproducing strain E. coli BL21 harboring the plasmid pAR1219 or purchased from Ambion (Austin, Tex.). For all buffers described, unless otherwise noted, final adjustment of pH is unnecessary.

Unnatural Amino Acids:

While most unnatural amino acids were purchased from commercial sources, other unnatural amino acids can be synthesized by known techniques. Tryptophan analogues were prepared using the method of Gilchrist et al. (1979) J. Chem. Soc. Chem. Commun. 1089-90. Tetrafluoroindole was prepared by the method of Rajh et al. (1979) Int. J. Pept. Protein Res. 14:68-79. 5, 7-Difluoroindole and 5,6,7-trifluoroindole were prepared by the reaction of CuI/dimethylformamide with the analogous 6-trimethylsilylacetylenylaniline.

Typically, the amino group was protected as the o-nitroveratryloxycarbonyl (NVOC) group, which was subsequently removed photochemically according to methods known in the art. However, for amino acids that have a photoreactive sidechain, an alternative, such as the 4-pentenoyl (4PO) group, a protecting group first described by Fraser-Reid, was used. Madsen et al. (1995) J. Org. Chem. 60:7920-7926; Lodder et al. (1997) J. Org. Chem. 62:778-779. We present here a representative procedure based on the unnatural amino acid (2-nitrophenyl)glycine (Npg), as described in England, et al. Proc. Natl. Acad. Sci. USA (in press).

N-4PO-D,L-(2-nitrophenyl)glycine. The unnatural amino acid D,L-(2-nitrophenylglycine) hydrochloride was prepared according to Davis et al. (1973) J. Med. Chem. 16:1043-1045; and Muralidharan et al. (1995) J. Photochem. Photobiol. B: Biol. 27:123-137. The amine was protected as the 4-pentenoyl (4PO) derivative as follows. To a room temperature solution of (2-nitrophenyl)glycine hydrochloride (82 mg, 0.35 mmol) in H₂O:dioxane (0.75 ml:0.5 ml) was added Na₂CO₃ (111 mg, 1.05 mmol) followed by a solution of 4-pentenoic anhydride (70.8 mg, 0.39 mmol) in dioxane (0.25 ml). After 3 hours the mixture was poured into saturated NaHSO₄ and extracted with CH₂Cl₂. The organic phase was dried over anhydrous Na₂SO₄ and concentrated in vacuo. The residual oil was purified by flash silica gel column chromatography to yield the title compound (73.2 mg, 75.2%) as a white solid. ¹H NMR (300 MHz, CD₃OD) δ 8.06 (dd, J=1.2, 8.1 Hz, 1H), 7.70 (ddd, J=1.2, 7.5, 7.5 Hz, 1H), 7.62-7.53 (m, 2H), 6.21 (s, 1H), 5.80 (m, 1H), 5.04-4.97 (m, 2H), 2.42-2.28 (m, 4H). HRMS calculated for C₁₃H₁₄N₂O₅ 279.0981, found 279.0992.

N-4PO-D,L-(2-nitrophenyl)glycinate cyanomethyl ester. The acid was activated as the cyanomethyl ester using standard methods known in the art (Robertson et al. (1989) Nucleic Acids Res. 17:9649-9660; Ellman et al. (1991) Meth. Enzym. 202:301-336). To a room temperature solution of the acid (63.2 mg, 0.23 mmol) in anhydrous DMF (1 ml) was added NEt3 (95 μl, 0.68 mmol) followed by ClCH₂CN (1 ml). After 16 hours the mixture was diluted with Et₂O, and extracted against H₂O. The organic phase was washed with saturated NaCl, dried over anhydrous Na₂SO₄, and concentrated in vacuo. The residual oil was purified by flash silica gel column chromatography to yield the title compound (62.6 mg, 85.8%) as a yellow solid. ¹H NMR (300 MHz, CDCl3) δ8.18 (dd, J=1.2, 8.1 Hz, 1H), 7.74-7.65 (m, 2H), 7.58 (ddd, J=1.8, 7.2, 8.4 Hz, 1H), 6.84 (d, J=7.8 Hz, 1H), 6.17 (d, J=6.2 Hz, 1H), 5.76 (m, 1H), 5.00 (dd, J=1.5, 15.6 Hz, 1H), 4.96 (dd, J=1.5, 9.9 Hz, 1H), 4.79 (d, J=15.6 Hz, 1H), 4.72 (d, J=15.6 Hz, 1H), 2.45-2.25 (m, 4H). HRMS calculated for C₁₆H₁₇N₃O₅ 317.1012, found 317.1004.

N-4PO-(2-nitrophenyl)glycine-dCA. The dinucleotide dCA was prepared as reported by Schultz (Id.) with the modifications described by Kearney et al. (1996) Mol. Pharmacol. 50:1401-1412. The cyanomethyl ester was then coupled to dCA as follows. To a room temperature solution of dCA (tetrabutylammonium salt, 20 mg, 16.6 μmol) in anhydrous DMF (400 μl) under argon was added N-4PO-D,L-(2-nitrophenyl)glycinate cyanomethyl ester (16.3 mg, 51.4 μmol). The solution was stirred for 1 hour and then quenched with 25 mM NH₄OAc, pH 4.5 (20 μl). The crude product was purified by reverse-phase semi-preparative HPLC (Whatman Partisil 10 ODS-3 column, 9.4 mm×50 cm), using a gradient from 25 mM NH₄OAc, pH 4.5 to CH₃CN. The appropriate fractions were combined and lyophilized. The resulting solid was redissolved in 10 mM HOAc/CH₃CN and lyophilized to afford 4PO-Npg-dCA (3.9 mg, 8.8%) as a pale yellow solid. ESI-MS M− 896 (31), [M−H]− 895 (100), calculated for C₃₂H₃₆N₁₀O17P₂ 896. The material was qualified by UV absorption (ε260≈37,000 M⁻¹ cm⁻¹).

Suppressor tRNA Design and Synthesis:

Suppressor tRNA which encode for the desired unnatural amino acid were made generally, for example, by the methods taught in Nowak et al. (1998) Methods in Enzymol. 293:515 and Petersson et al. RNA 2002 April;8(4):542-7. The following procedure was followed for the suppressor tRNA THG73. The gene for T. thermophila tRNAGln CUA G73, flanked by an upstream T7 promoter and a downstream Fok I restriction site, and lacking CA at positions 75 and 76, was constructed from eight overlapping DNA oligonucleotides (SEQ ID NOs: 1-8) and cloned into the pUC19 vector.

| | |
|---|---|
| SEQ ID NO:1 | 5′-AATTCGTAATACGACTCTACTATAGGTTCTATAG-3′ |
| SEQ ID NO:2 | 3′-GCATTATGCTGAGTGATATCCAAGA-5′ |
| SEQ ID NO:3 | 5′-TATAGCGGTTAGTACTGGGGACTCTAAA-3′ |
| SEQ ID NO:4 | 3′-TATCATATCGCCAATCATGACCCCTGAG-5′ |
| SEQ ID NO:5 | 5′-TCCCTTGACCTGGGTTCG-3′ |
| SEQ ID NO:6 | 3′-ATTTAGGGAACTGGACCC-5′ |
| SEQ ID NO:7 | 5′-AATCCCAGTAGGACCGCCATGAGACCCATCCG-3′ |
| SEQ ID NO:8 | 3′-AGCTTAGGGTCATCCTGGCGGTACTCTGGGTAGGCCTAG-5′ |

Digestion of the resulting plasmid (pTHG73) with Fok I gave a linearized DNA template corresponding to the tRNA transcript, minus the CA at positions 75 and 76. In vitro transcription of Fok I linearized pTHG73 was performed as described by Sampson et al. (1988) Proc. Natl. Acad. Sci. 85:1033. The 74-nucleotide tRNA transcript, tRNA- THG73 (minus CA) was purified to single nucleotide resolution by denaturing polyacrylamide electrophoresis and then quantitated by ultraviolet absorption.

Chemical acylation of tRNAs and removal of protecting groups:

The α-NH2-protected dCA-amino acids or dCA were enzymatically coupled to the THG73 FokI runoff transcripts using T4 RNA ligase to form a full-length chemically charged α-NH2-protected aminoacyl-THG73 or a full-length but unacylated THG73-dCA.

Prior to ligation, 10 μl of THG73 (1 μg/μl in water) was mixed with 5 μl of 10 mM HEPES, pH 7.5. This tRNA/HEPES premix was heated at 95° C. for 3 min and allowed to cool slowly to 37° C.

After incubation at 37° C. for 2 hours, DEPC-H₂O (52 μl) and 3M sodium acetate, pH 5.0 (8 μl), were added and the reaction mixture was extracted once with an equal volume of phenol (saturated with 300 mM sodium acetate, pH 5.0):CHCl₃:isoamyl alcohol (25:24:1) and once with an equal volume of CHCl₃:isoamyl alcohol (24:1) then precipitated with 2.5 volumes of cold ethanol at −20° C. The mixture was centrifuged at 14,000 rpm at 4° C. for 15 min, and the pellet was washed with cold 70% (v/v) ethanol, dried under vacuum, and resuspended in 7 μl 1 mM sodium acetate, pH 5.0. The amount of α-NH2-protected amino acyl-THG73 was quantified by measuring A260, and the concentration adjusted to 1 μg/μl with 1 mM sodium acetate pH 5.0.

The ligation efficiency was determined from analytical PAGE. The α-NH2-protected amino acyl-tRNA partially hydrolyzes under typical gel conditions, leading to multiple bands, so the ligated tRNA was deprotected prior to loading. Such deprotected tRNAs immediately hydrolyze on loading. Typically, 1 μg of ligated tRNA in 10 μl BPB/XC buffer was loaded onto the gel, and 1 μg of unligated tRNA was run as a size standard. The ligation efficiency was determined from the relative intensities of the bands corresponding to ligated tRNA (76 bases) and unligated tRNA (74 bases).

Generation of mRNA:

For the nonsense codon suppression method, it is desirable to have the gene of interest in a high-expression plasmid, so that functional responses in oocytes may be observed 1-2 days after injection. Among other considerations, this minimizes distortions due to eventual reacylation of the suppressor tRNA. A high-expression plasmid was generated by modifying the multiple cloning region of pBluescript SK+ (Nowak et al. (1998)). At the 5′ end, an alfalfa mosaic virus (AMV) sequence was inserted, and at the 3′ end a poly(A) tail was added, providing the plasmid pAMV-PA. mRNA transcripts containing the AMV region bind the ribosomal complex with high affinity, leading to 30-fold increase in protein synthesis. Including a 3′poly (A) tail was shown to increase mRNA half-life, therefore increasing the amount of protein synthesized. The gene of interest was subcloned into pAMV-PA such that the AMV region is immediately 5′ of the ATG start codon of the gene (i.e. the 5′ untranslated region of the gene was completely removed). The plasmid pAMV-PA was made available from C. Labaraca at Caltech (Nowak et al. (1998)).

TAG stop codons at positions where unnatural amino acid incorporation is desired were produced by site directed mutagenesis. Suitable site-directed mutagenesis methods used to create stop codons at the desired positions include: the Transformer kit (Clontech, Palo Alto, Calif.), the Altered Sites kit (Stratagene, La Jolla, Calif.), and standard polymerase chain reaction (PCR) cassette mutagenesis procedures. With the first two methods, a small region of the mutant plasmid (400-600 base pairs) was subcloned into the original plasmid. With all methods, the inserted DNA regions were checked by automated sequencing over the ligated sites. The pAMV-PA plasmid constructs were linearized with NotI and mRNA transcripts were generated using the mMessage mMachine T7 RNA polymerase kit (Ambion, Austin, Tex.).

Oocytes—Preparation and Injection:

Oocytes were removed from Xenopus laevis using techniques known in the art, such as Quick et al. (1994) J. Biol. Chem. 269(48):30164-72. Methods for expression of excitability proteins in Xenopus Oocytes were found in Ion Channels ofExcitable Cells. (Narahashi, T., ed.), pp 261-279, Academic Press, San Diego, Calif., USA. Oocytes were maintained at 18° C. in ND96 solution consisting of 96 mM NaCl, 2 mM KCl, 1 mM MgCl₂, 1.8 mM CaCl₂, 5 mM HEPES (pH 7.5), supplemented with sodium pyruvate (2.5 mM), gentamicin (50 μg/ml), theophylline (0.6 mM) and horse serum (5%). Prior to injection, the NVOC-aminoacyl-tRNA (1 μg/μl) in I mM NaOAc (pH 5.0) was deprotected by irradiating for 5 min. with a 1000 W xenon arc lamp (Oriel) operating at 600 W equipped with WG-335 and UG-11 filters (Schott). The deprotected aminoacyl-tRNA was mixed 1:1 with a water solution of the desired mRNA. Oocytes were injected with 50 nl of a mixture containing 25-50 ng aminoacyl-tRNA and 12.5-18 ng of total receptor mRNA (ratio of 20:1:1:1 for α:β:γ.δ subunits).

Electrophysiology:

Electrophysiological measurements will be carried out 18-30 hr after oocyte injection by two electrode voltage clamp using the OpusXpress (Axon Instruments, Union City, Calif.). The OpusXpress has the capacity to record from 8 oocytes in parallel. Further, perfusion of the oocytes is fully automated. Oocytes will be continuously bathed in ND96 (5 mM HEPES, pH 7.4, 96 mM NaCl, 2 mM KCl and 1 mM MgCl₂ (CaCl₂ will be omitted to prevent activation of Ca⁺⁺-dependent Cl⁻ currents). Changes in currents due to ligand interactions will be recorded by automated bath application of the desired compounds at various concentrations Agonist-induced currents will be recorded by automated bath application of the desired agonist concentration. Measurements will be made at a holding potential of −80 mV. To minimize distortion of current responses due to acute receptor desensitization agonist applications will be at five minute intervals to allow for receptors to recover. This has been previously shown to prevent distorting effects associated with nAChR desensitization in oocytes It may be necessary to increase the wash period between agonist applications, particularly at agonist concentrations >EC50 values.

Development of Receptophore Model:

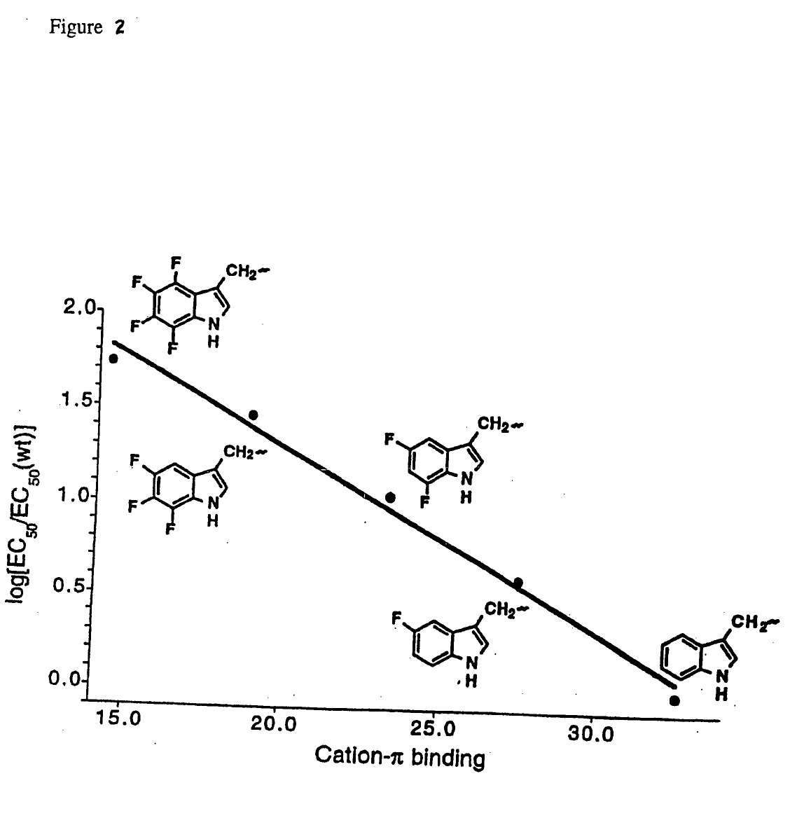

Dose-response curves were fitted to the Hill equation for the unaltered receptor (WT) and for unnatural amino acid substitutions at α-Trp 149. Substitutions include 5-F-Trp, 5,7-F₂-Trp, 5,6,7-F₃-Trp and 4,5,6,7-F₄-Trp. The log[EC₅₀/EC₅₀ (WT)] for each substitution and for the unaltered receptor was plotted vs. cation-π binding ability of each fluorinated trp derivative. Cation-π binding ability for trp and the fluorinated derivatives were predicted using ab initio quantum mechanical calculations (Mecozzi et al. (1996) J. Amer. Chem. Soc. 118:2307-2308; and Mecozzi et al. (1996) Proc. Natl. Acad. Sci. 93:10566-10571). Data fit the line y=3.2−0.096x, with a correlation coefficient r=0.99. See FIG. 2. These data are consistent with a cation-π bond between α-trp 149 and the quaternary ammonium of acetylcholine in the bound position because each substitution's EC₅₀ value corresponds well with the predicted loss in binding energy due to the substitution.

### EXAMPLE 2

Unnatural amino acids were incorporated into the nAChR using in vivo nonsense suppression methods, and mutant receptors were evaluated electrophysiologically (Dougherty, D. A. (2000) Curr. Opin. Chem. Biol. 4:645-652). The structures and electrostatic potential surfaces for these cationic agonists was positive everywhere.

In studies of weak agonists and/or receptors with diminished binding capability, it is necessary to introduce another mutation that independently decreases EC₅₀. This was accomplished via a Leu-to-Ser mutation in the β subunit at a site known as 9′ in the M2 transmembrane region of the receptor. This M2-β9′ site is almost 50 Å from the binding site, and previous work has shown that a Leu9′Ser mutation lowers the EC₅₀ by a factor of roughly 10 without altering trends in EC₅₀ values (Beene, D. L., et al. (2002) Biochem. 41:10262-9; and Kearney, P. C., et al. (1996) Mil. Pharmacol. 50:1401-1412). Measurements of EC₅₀ represent a functional assay; all mutant receptors reported here are fully functioning LGICs. It is important to note that the EC₅₀ value is not a binding constant, but a composite of equilibria for both binding and gating.

Epibatidine Binds with a Potent Cation-π Interaction at Trp a149.

The existence of a cation-π interaction between Epi and Trp α149 was evaluated via the incorporation of a series of fluorinated Trp derivatives (5-F-Trp, 5, 7-F2-Trp, 5, 6, 7-F₃-Trp and 4, 5, 6, 7-F₄-Trp). The EC₅₀ values of the wild type and mutant receptors are shown in Table 1. Attempts to record dose-response relations from 4, 5, 6, 7-F4-Trp at α149 were unsuccessful, because this mutant required Epi concentrations above 100 μM. At these concentrations Epi becomes an effective open channel blocker (Prince, R. J. & Sine, S. M. (1998) Biophys. J. 75:1817-27), confounding efforts to obtain an accurate dose-response curve. A clear trend can be seen in the data of Table 1 in which each additional fluorine produces an increase in EC₅₀.

TABLE 1
Mutations Testing Cation-π Interactions at α149

| | Trp | F-Trp | F₂-Trp | F₃-Trp |
|---|---|---|---|---|
| Epi* | 0.72 ± 0.08^{‡} | 3.5 ± 0.1 | 7.5 ± 0.4 | 15 ± 1 |
| Cation-π^{†} | 32.6 | 27.5 | 23.3 | 18.9 |

*EC₅₀ (μM) ± standard error of the mean. The receptor has a Leu9′Ser mutation in M2 of the β subunit.
^{†}Zhong et al. (1998) supra. Binding energy of a probe cation (Na⁺) to the ring in kcal/mol.
^{‡}Rescue of wild type by nonsense suppression.

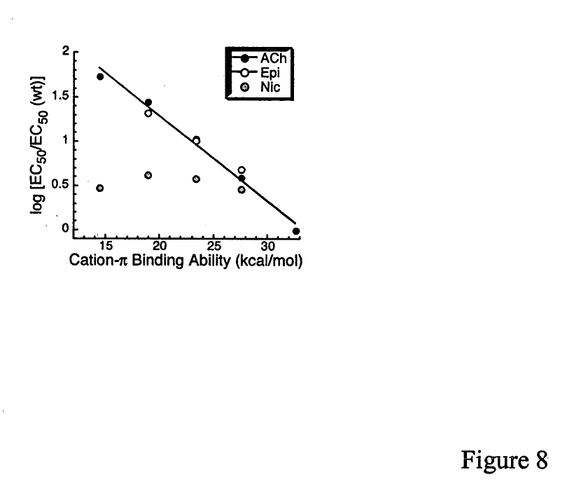

The measure for the cation-π binding ability of the fluorinated Trp derivatives is the calculated binding energy of a generic probe cation (Na+) to the corresponding substituted indole (Zhong, W., et al. (1998) Proc. Natl. Acad. Sci. USA 95:12088-93; and Beene, D. L., et al. supra). This method provides a convenient way to express the clear trend in the dose-response data in a quantitative way. A “fluorination plot” of the logarithmic ratio of the mutant EC₅₀ to the wild type EC₅₀ versus the cation-π binding ability for Trp α149 reveals a linear relationship (FIG. 8). These data demonstrate that the secondary ammonium group of Epi makes a cation-π interaction with Trp α149 in the muscle-type nAChR.

Nicotine and Epibatidine Hydrogen Bond to the Carbonyl Oxygen of Trp α149.

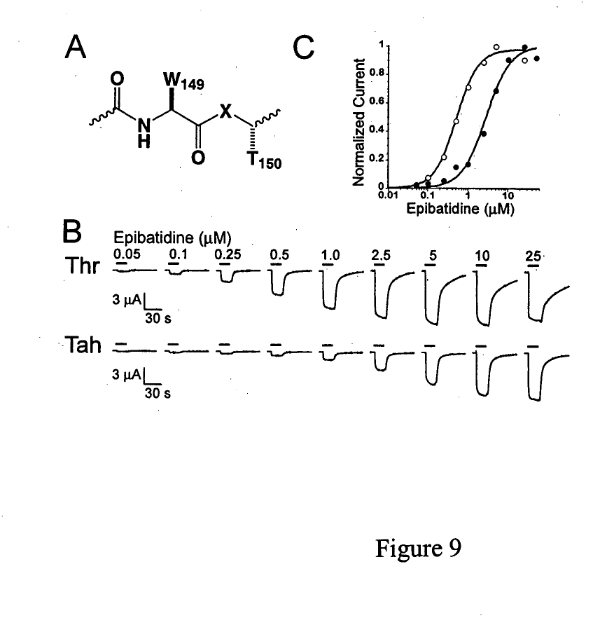

The reported crystal structure of AChBP with Nic bound indicated a hydrogen bond between the pyrrolidine N⁺—H of Nic and the backbone carbonyl of Trp α149 (Celie, P. et al. (2004) Neuron 41:907-914), an interaction that had been anticipated by several modeling studies. To evaluate this possibility, the backbone amide at this position was converted to an ester by replacing Thr α150 with the analog α-hydroxy threonine (Tah) using the nonsense suppression methodology (FIG. 9a). Converting an amide carbonyl to an ester carbonyl weakens the hydrogen bonding ability of the oxygen, an effect that has been estimated to be worth ˜0.9 kcal/ml (Koh, J. T., et al. (1997) Biochem. 36:11314-22).

The results of the incorporation of Tah at α150 are shown in Table 2. Upon ester substitution, the EC₅₀ for Nic increases 1.6 fold. The change is larger for the more potent agonist Epi; conversion of the backbone carbonyl of Trp α149 to an ester leads to a 3.7-fold increase in EC₅₀ (FIG. 9). In contrast, ACh, lacking a proton at the cationic center, shows a 3.3 fold decrease in EC₅₀. These results further highlight the distinction between nicotinic and cholinergic agonists.

TABLE 2
Mutations Testing H-bond Interactions at α150*

| Agonist | Thr^{†} | Tah | Tah/Thr |
|---|---|---|---|
| ACh | 0.83 ± 0.04 | 0.25 ± 0.01 | 0.31 |
| Nic | 57 ± 2 | 92 ± 4 | 1.6 |
| Epi | 0.60 ± 0.04 | 2.2 ± 0.2 | 3.7 |

*EC₅₀ (μM) ± standard error of the mean. The receptor has a Leu9′Ser mutation in M2 of the β subunit.
^{†}Rescue of wild type by nonsense suppression.

Computational Modeling.

Computational modeling was used to understand the variations in binding properties among the three agonists. Focusing on the interactions with Trp α149, the ligands were docked using ab initio (HF/6-31G) calculations taking into account both the cation-π interaction and the carbonyl hydrogen bond. Initial tryptophan and ligand coordinates were taken from the AChBP-based homology models of Le Novere, N., et al. (2002) Proc. Natl. Acad. Sci. 99:3210-3215. Geometry optimizations, counterpoise corrections, and zero point energy corrections were all performed in the gas phase. The optimized geometries for free ACh and Nic are in keeping with previous calculations at higher levels of theory and with solution NMR studies, in that bent “tg” structures are favored (Elmore, D. E. & Dougherty, D. A. (2000) J. Org. Chem. 65:742-747; and Partington, P., et al. (1972) Mol. Pharmacol. 8:269-77). The calculated binding energies were consistent with those from previous computational studies of metal binding complexes with both cation-π and cation-carbonyl interactions (Siu F. M., et al. (2004) Chem. Eur. J. 10:1966-1976) and studies of hydrogen bonds to protonated Nic (Graton, J., et al. (2003) J. Org. Chem. 68:8208-8221; and Graton, J., et al. (2003) J. Am. Chem. Soc. 125:5988-97).

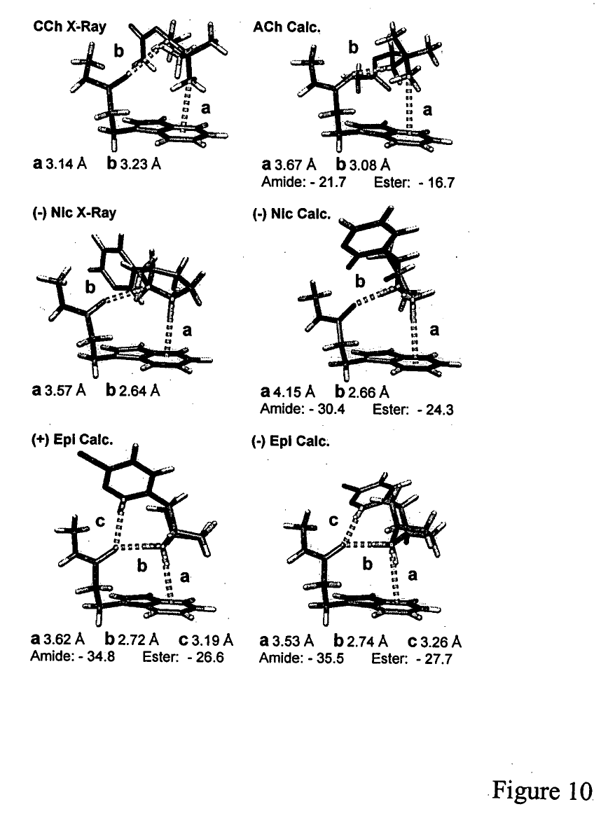

The calculated binding energies are summarized in FIG. 10. Experimentally, the EC₅₀s of (+) and (−) Epi were nearly identical for a given acetylcholine receptor subtype (Spande, T. F., et al. (1992) J. Am. Chem. Soc. 114:3475-3478), and the calculated binding energies and the key geometrical parameters (FIG. 10) were very similar for the two enantiomers. Epibatidine bound the amide more strongly than Nic by ˜5 kcal/mol. Conversion of the Trp α149 amide to an ester weakened the binding interactions to both Epi and Nic. The calculated energetic effect of ester conversion was larger for Epi than for Nic (8 kcal/mol vs. 6 kal/mol). Using the PCM solvation model (Cossi, J., et al. (1996), supra.), the interactions in solvents of differing polarity (Table 3) were studied. In each solvent, Epi favored amide binding over ester binding to a greater degree than Nic. The changes in hydrogen bonding energies observed in different solvent systems were consistent with similar calculations published by Cannizzaro, C. E. & Houk, K. N. (2002) J. Am. Chem. Soc. 124:7163-9.

TABLE 3
Solvent Effects on Binding Energy Differences*

Ester Binding Energy -
Amide Binding Energy (kcal/mol)

| Agonist | Gas | THF | Ethanol | Water |
|---|---|---|---|---|
| ACh | 5.0 | 0.6 | −1.7 | −2.0 |
| Nic | 6.1 | 3.1 | 1.2 | −0.8 |
| Epi^{†} | 8.0 | 7.0 | 5.0 | 4.7 |

*ε(THF) = 7.6, ε(ethanol) = 24.3, ε(water) = 78.5.
^{†}Average of energies for epibatidine enantiomers.

The geometries of FIG. 10 are consistent with the energetic trends observed. The cation-π interaction is expected to be much stronger for Epi than for Nic. The calculated N⁺ to π-centroid distance is substantially shorter for Epi (a in FIG. 10). In addition, Epi points an N⁺—H cationic center towards the Trp indole ring, vs. the N⁺CH₂—H center of Nic (FIG. 10). The cationic center of Epi has a much more positive electrostatic potential than that of Nic (+139 kcal/mol for Epi, +112 for Nic). These potentials, indicators of cation-π binding strength, are consistent with the experimental observation that epibatidine has a much stronger cation-π interaction than Nic.

Nicotine and Epi make significant hydrogen bonds to the Trp α149 carbonyl oxygen with an N⁺—H group (b in FIG. 10). The geometrical parameters for interaction b with the two agonists are very similar, suggesting the two hydrogen bonds are comparably strong. In addition, the calculations suggest a second, previously unanticipated interaction between the C_{aromatic}—H of the carbon adjacent to the pyridine N and the same carbonyl (c in FIG. 10). Based on both the distance (c in FIG. 10) and angle (C—H—O=168° in Epi vs. 145° in Nic), one would expect the C_{aromatic}H•••O═C interaction to be stronger for Epi interaction than for Nic.

A number of studies have identified key interactions that lead to the binding of small molecules at the agonist-binding site of nAChRs (Schmitt, J. D. (2000) Curr. Med. Chem. 7:749-800). The field was dramatically altered with the appearance of the crystal structure of the ACh binding protein. ACHBP is not the nAChR, however, it is a small, soluble protein secreted from the glial cells of a snail, and it is <25% identical to its closest relative in the nAChR family, α7 (Brejc, K., et al., (2001) Nature 411:269-76).

Previously, it was observed that Nic and ACh use different noncovalent interactions to bind the muscle-type nAChR. ACh forms a strong cation-π interaction with Trp α149; Nic does not. Although known as the nicotinic receptor, the form found in the peripheral nervous system, it is relatively insensitive to Nic. At this muscle-type receptor ACh is over 70-fold more potent than Nic. The behavioral and addictive effects of Nic arise exclusively from interactions with one or more neuronal subtypes of nAChR found in the central nervous system, where Nic and ACh are generally comparably potent. A probe for a nicotinic-type agonist that is potent at the muscle receptor, was needed, and Epi was the logical choice. This alkaloid natural possesses potent analgesic properties (Spande, T. F., et al., (1992) supra.), and has served as a lead compound for a number of pharmaceutical programs targeted at the nAChR (Dukat, M. & Glennon, R. A. (2003) Cell Mol. Neurobiol. 23:365-78). In this example, two specific interactions that distinguish among the three agonists (ACh, Nic, and Epi) were studied.

First, Epi made a strong cation-π interaction with Trp α149 of the muscle-type nAChR. This result contrasted sharply to Nic, and this observation helped to explain the much higher affinity of Epi for this receptor relative to Nic. The apparent magnitudes of the cation-π interactions, indicated by the slopes of the fluorination plots in FIG. 8, were comparable for ACh and Epi. This similarity was surprising, considering the cationic centers of the two agonists are chemically quite different (quaternary ammonium for ACh; protonated secondary ammonium for Epi). The computer modeling summarized in FIG. 10 rationalized the observed cation-π binding behavior. Epi, like ACh, made much closer contact with the indole ring than Nic. Both the interaction distance (a in FIG. 10) and the electrostatic potential on the cationic hydrogen N⁺—H in Epi; vs. N⁺CH₂—H in Nic favored the cation-π interaction in Epi over Nic.

The second discriminator probed was hydrogen bonding. A newer crystal structure of the AChBP included Nic at the binding site (Celie, P. H. N., et al. (2004) supra.). The structure confirmed the existence of a hydrogen bond between Nic and the backbone carbonyl of Trp α149, an interaction anticipated by modeling studies. In efforts to probe this non-covalent interaction, the effects of decreasing the hydrogen bond acceptor ability of the backbone carbonyl of Trp α149 were studied. In such studies, the clear distinction between ACh and nicotinic agonists was strengthened. Nic and Epi, containing a tertiary and secondary cationic center respectively, both showed increases in EC₅₀ compared to the native receptor in response to the amide-to-ester modification (Table 2). The effect was larger with the more potent agonists, Epi. Thus, the experimental data supported that Nic and Epi interact with the nAChR through a hydrogen bond with the backbone carbonyl of Trp α149.

ACh, with a quaternary cationic center that cannot make a conventional hydrogen bond, shows a decrease in EC₅₀ at the ester-containing receptor compared to the native receptor even though it was anticipated that the binding of ACh would be unaffected by such a subtle change. The origin of this effect is presently unclear, however, two possibilities are listed below.

In the recently reported crystal structure of AChBP bindng to carbamylcholine (CCh), a cholinergic analogue of ACh, the backbone carbonyl oxygen of interest makes contact with a CH₂ group adjacent to the N⁺(CH₃)₃ group (CCh: NH₂C(O)OCH₂CH₂N⁺(CH₃)₃). This N⁺Ch₂ carries a significant positive charge, like the N⁺CH₃ groups, and so a favorable electrostatic interaction is possible. This interaction with CCh would be much weaker than the N⁺—H hydrogen bonds of Nic and Epi, but perhaps not negligible. Interestingly, Sixma and coworkers noted that the binding of CCh to ACHBP is less enthalpically favorable than that of Nic. They attribute this observation to the net unfavorable burial of the carbonyl oxygen by CCh—the weak interaction with the CH₂ group cannot compensate for the loss of hydrogen bonding, presumably to water molecules. With Nic, a strong hydrogen bond compensates this desolvation penalty more effectively.

The relatively simple model calculations conducted recapitulate this effect. In the gas phase, it is better to bind to the backbone amide than the ester for all three agonists. However, as solvation is introduced, the trend is reversed (Table 3). When a solvent of moderate polarity such as ethanol is used, ACh prefers the ester backbone, while Nic and Epi prefer the amide. The ethanol environment is defined in these calculations by a dielectric constant of 24.3. Two lines of evidence indicate that this is a reasonable estimate of the effective dielectric of the binding pocket of the AChBP or nAChR. First, it is consistent with previous experimental measurements of a perturbed local pKₐ in the nAChR binding site (Peterson, E. J., et al. (2002) J. Am. Chem. Soc. 124:12662-3). Second, calculations of the solvent accessible surface area of the binding site residues show that Trp 149 is 11% solvent-accessible. A moderate dielectric of 24.3 seems reasonable for the partially-exposed binding site. Thus, it may be that the EC₅₀ for ACh decreases when the ester is introduced because the desolvation penalty of the ester carbonyl oxygen is less severe than the amide.

A second possible explanation is that highly conserved Asp α89 (Asp 85 AChBP numbering) makes a number of significant contacts with nearby residues, suggesting it plays a key structural role in shaping the agonist binding site (Brejc, K., et al. (2004) supra.). One such interaction is a hydrogen bond between the Asp α89 carboxylate side chain and the NH group of the backbone amide of Trp α149. The amide-to-ester mutation eliminates the NH and so removes this interaction. A possible outcome would be a structural change that would affect gating, biasing the conformational change in the direction of the open channel.

Regardless of the origin of the effect, it is reasonable to propose that the effect of ester substitution seen with ACh can be considered as the “background” for the Thrl150Tah mutation. That is, if the magnitude of the cholinergic N⁺CH₂•••O═C interaction is small, then both the desolvation and gating effects proposed are “generic” and should occur with all agonists. Therefore, the changes in EC₅₀ measured for Nic or Epi actually represent the product of two terms: a generic 3.3-fold decrease evidenced by ACh, and a term specific to Nic or Epi. The drop in hydrogen bonding strength is calculated to be 1.6*3.3 or ˜5-fold for Nic, and 3.7*3.3 or ˜12-fold for Epi. Energetically, these factors correspond to 1.0 and 1.5 kcal/mol, respectively, which is quite consistent with the modulation of a hydrogen bond.

The larger amide/ester effect seen for Epi vs. Nic suggests a stronger N⁺—H•••O═C hydrogen bond in the former. However, these hydrogen bonds (b in FIG. 10) are geometrically very similar in the two complexes, suggesting that they are of comparable strengths. An alternative rationalization invoking the novel C_{aromatic}—H hydrogen bond revealed by modeling studies has been proposed. Aromatic hydrogens intrinsically carry a significant positive electrostatic potential (+18 kcal/mol in benzene). This effect is amplified when the carbon is ortho to a pyridine-type N (+24 kcal/mol in pyridine) and meta to an electron-withdrawing C1 (+31 kcal/mol in 2-chloropyridine.). Thus, interaction c should be energetically significant. Geometrically, the C_{aromatic}—H hydrogen bond to the carbonyl (c in FIG. 10) is much tighter and better aligned for Epi than Nic. The computations thus suggest that it is this unconventional hydrogen bond (c), rather than the anticipated hydrogen bond (b), that rationalizes the slightly greater response of Epi vs. Nic to the backbone change. Thus, the small structural differences between Epi and Nic nicely explain their differing affinities. The secondary ammonium of Epi provides two N⁺—Hs that can undergo strong electrostatic interactions—a cation-π interaction and a hydrogen bond to a carbonyl. The tertiary ammonium of Nic can only make the hydrogen bond. Second, the slightly different positioning of the pyridine group in Epi allows for a more favorable C_{aromatic}—H•••O═C hydrogen bond than for Nic.

In summary, a combination of unnatural amino acids mutagenesis and computer modeling has led to the following conclusions. The nicotinic agonists Nic and Epi both experience a favorable hydrogen bonding interaction with the carbonyl of Trp α149, which is qualitatively distinct from the interaction (if any) of ACh with this group. Also, Epi is a much more potent agonist than Nic at the muscle-type nAChR because, along with hydrogen bonding, Epi experiences a cation-π interaction comparable to that seen with ACh, while Nic does not. In addition, Epi picks up a subtle C_{aromatic}—H•••O═C hydrogen bond that Nic does not.

Materials and Methods

Preparation of α-hydroxythreonine (Tah).

α-hydroxythreonine (Tah) (2R, 3S-dihydroxy-butanoate) cyanomethyl ester was synthesized according to previously published methods (Servi, S. (1985) J. Org. Chem., 50:5865-5867; and England, P. M., et al. (1999) Tetrahedron Lett. 40:6189-6192).

Electrophysiology.

Stage VI oocytes of Xenopus laevis were employed. Oocyte recordings were made 24 to 48 h post injection in two-electrode voltage clamp mode using the OpusXpress™ 6000A (Axon Instruments, Union City, Calif.). Oocytes were superfused with Ca²⁺-free ND96 solution at flow rates of 1 ml/min, 4 ml/min during drug application and 3 ml/min during wash. Holding potentials were −60 mV. Data were sampled at 125 Hz and filtered at 50 Hz. Drug applications were 15 s in duration. Agonists were purchased from Sigma/Aldrich/RBI: ([−] nicotine tartrate), (acetylcholine chloride) and ([±] epibatidine). Epi was also purchased from Tocris ([±] epibatidine). All drugs were prepared in sterile ddi water for dilution into calcium-free ND96. Dose-response data were obtained for a minimum of 10 concentrations of agonists and for a minimum of 7 cells. Dose-response relations were fitted to the Hill equation to determine EC₅₀ and Hill coefficient. EC₅₀ for individual oocytes were averaged to obtain the reported values.

Unnatural Amino Acid Suppression.

Synthetic amino acids and α-hydroxy acids were conjugated to the dinucleotide dCA and ligated to truncated 74 nt tRNA as previously described in England, P. M., et al. (1999) supra; and Nowak, M. W., et al. (1998) Methods Enzymol. 293:504-529. Deprotection of amino acyl tRNA was carried out by photolysis immediately prior to co-injection with mRNA, as described in Nowak M. W., et al. (1998) supra; and Li, L. T., et al. (2001) Chem. Biol. 8:47-58. Typically, 25 ng of tRNA were injected per oocyte along with mRNA in a total volume of 50 nl/cell. mRNA was prepared by in vitro runoff transcription using the Ambion (Austin, Tex.) mMessage mMachine kit. Mutation to the amber stop codon at the site of interest was accomplished by standard means and was verified by sequencing through both strands. For nAChR suppression, a total of 4.0 ng of mRNA was injected in the subunit ratio of 10:1:1:1 α:β:γ:δ. In all cases, the β subunit contained a Leu9′Ser mutation, as discussed above. Mouse muscle embryonic nAChR in the pAMV vector was used. In addition, the α subunits contain an HA epitope in the M3-M4 cytoplasmic loop for biochemical studies. Control experiments showed a negligible effect of this epitope on EC₅₀. As a negative control for suppression, truncated 74 nt or truncated tRNA ligated to dCA was co-injected with mRNA in the same manner as fully charged tRNA. No current was observed from these negative controls. The positive control for suppression involved wild-type recovery by co-injection with 74 nt tRNA ligated to dCA-Thr or dCA-Trp. The dose-response data were indistinguishable from injection of wild-type mRNA alone.

Computation.

Acetylcholine, (−) nicotine, (+) epibatidine, (−) epibatidine, 3-(1H-Indol-3-yl)-N-methyl-propionamide, 3-(1H-Indol-3-yl)-O-methyl-propionate and the hydrogen-bonded complexes shown in FIG. 10 were optimized at the HF/6-31G level of theory. For the acetylcholine, (−) nicotine, and (−) epibatidine complexes, the starting coordinates of the ligand and Trp 147 (α7 numbering) were taken from the docked structures of Changeux and coworkers available at http://www.pasteur.fr/recherche/banques/LGIC/LGIC.html. The optimized geometries were fully characterized as minimal by frequency analysis. Energies were calculated at the HF/6-31G level. Basis set superposition error (BSSE) corrections were determined in the gas phase at HF/6-31G level, using the counterpoise correction method of Boys, S. F. & Bernardi, F. (1970) Mol. Phys. 19:553-554. Zero point energy (ZPE) corrections were included by scaling the ZPE correction given in the HF/6-31G level frequency calculation by the factor of 0.9135 given by Foresman, J. B. & Frisch, E. (1996) Exploring Chemistry With Electronic Structure Methods (Gaussian, Inc., Pittsburgh, Pa.). All calculations were carried out with the Gaussian 98 program, (M. J., Trucks, et al. (1998) (Gaussian, Inc., Pittsburgh Pa.)). Binding energies were determined by comparing the BSSE- and ZPE-corrected energies of the separately optimized ligand and tryptophan analog to the energy of the complex. Solvent effects were added to the gas phase structures using the polarizable continuum model (PCM) self-consistent reaction field (Cossi, M., et al. (1996) Chem. Phys. Lett. 225:327-335) with ε(THF)=7.6, ε(EtOH) =24.3, and ε(H2O)=78.5. The optimized geometries are reported within.

Electrostatic potential surfaces were created with Molekel, available at www.cscs.ch/molekel/ Flukiger, P., et al. (2000) Swiss Center for Scientific Computing, Manno, Switzerland. The electrostatic potential for each structure was mapped onto a total electron density surface contour at 0.002 e/Å³. Benzene, pyridine, and 2-chloropyridine were also optimized at the HF/6-13G level of theory and their electrostatic potential surfaces were calculated.

All references cited herein are incorporated by reference in their entirety.

While the invention has been described in conjunction with examples thereof, it is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention. Thus, the invention is intended to be defined not by the above description, but by the following claims and their equivalents.

1. A method for determining the nature of a compound's interaction with a receptor comprising:
a. incorporating unnatural amino acids into binding sites and regulatory sites of the receptor, resulting in an altered receptor;
b. measuring the compound's ability to bind to the altered receptor; and
c. comparing the results of step (b) to the same compound's ability to bind to an unaltered receptor.

2. The method of claim 1 wherein said unnatural amino acids are selected from the following Formula (I): 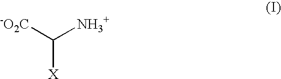
where X is selected from the group consisting of:

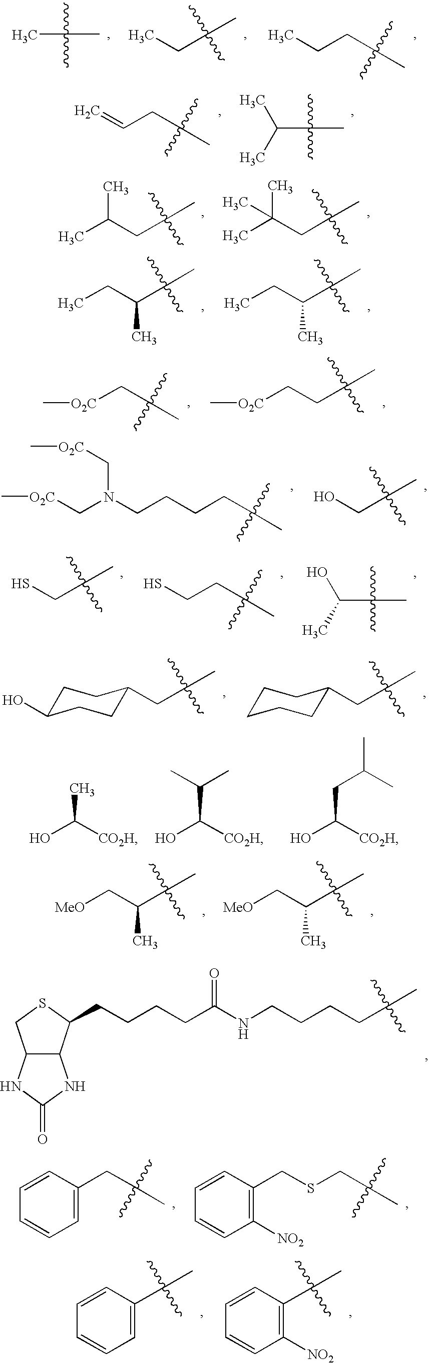

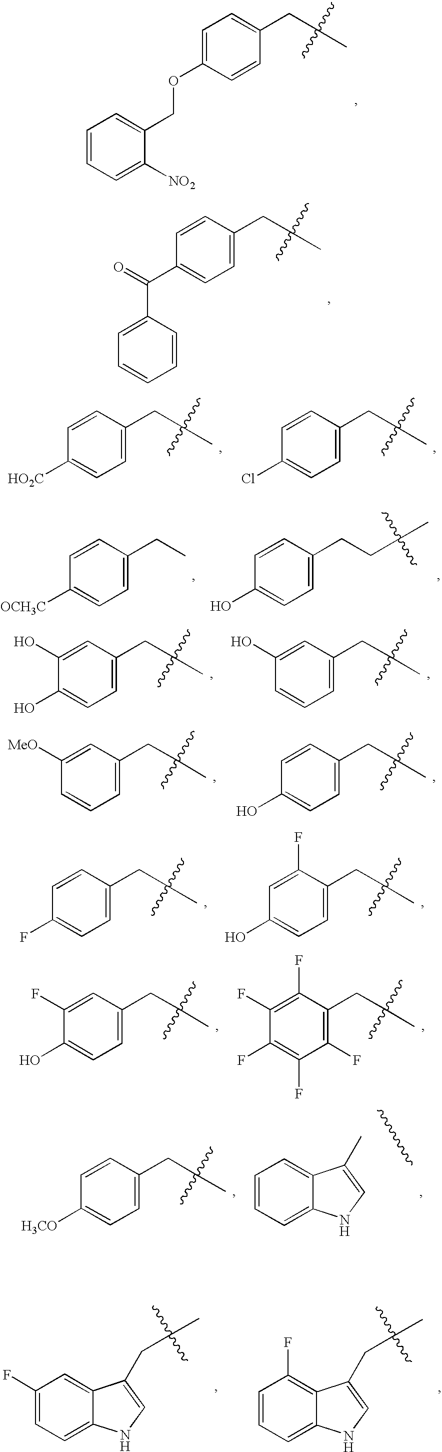

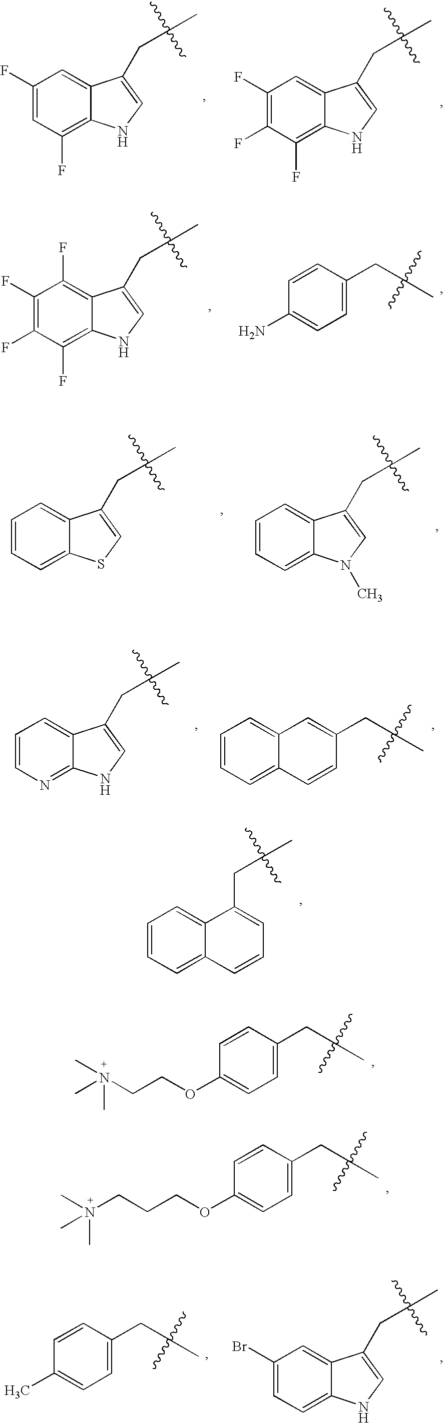

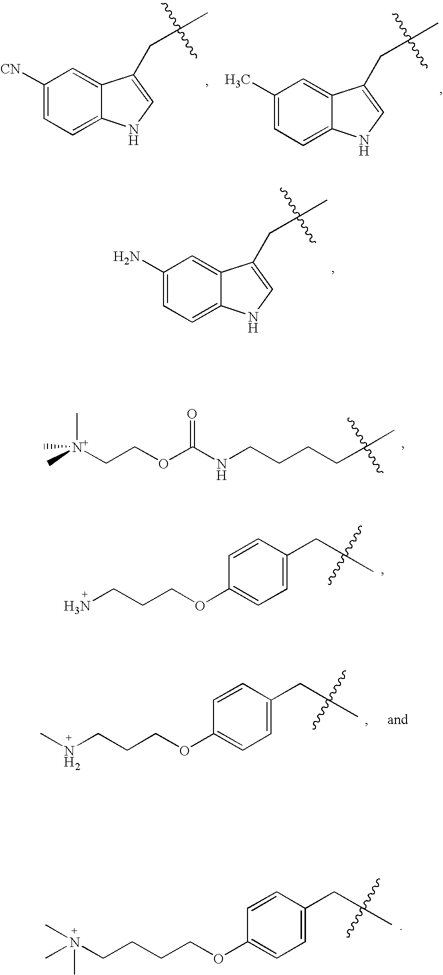

3. The method of claim 1 wherein the unnatural amino acids are selected from the following Formula (II): 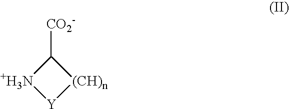
wherein: Y is CH₂, (CH)ₙ, N, O, or S, and n is 1 or 2.

4. The method of claim 3 wherein the unnatural amino acid is selected from the group consisting of:

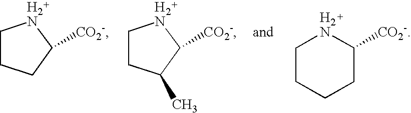

5. The method of claim 1 wherein the receptor is expressed in Xenopus oocytes.

6. A method of altering a compound so that it interacts with its receptor to achieve desired ligand activity:
a. determining the nature of the compound's interaction with the receptor;
b. analyzing how and where the compound interacts with the receptor;
c. based on the analysis in step (b), chemically modifying the compound to achieve desired ligand activity.

7. A screening method comprising a GPCR or LGIC membrane protein receptor which has been modified at the binding or regulatory site to replace native amino acids with unnatural amino acids.

8. The screening method of claim 7 wherein the native amino acids to be replaced are selected from the group consisting of any of the 20 naturally occurring amino side chains.

9. The screening method of claim 7 wherein said unnatural amino acids are selected from the following Formula (I): 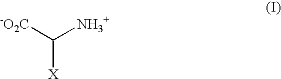
where X is selected from the group consisting of:

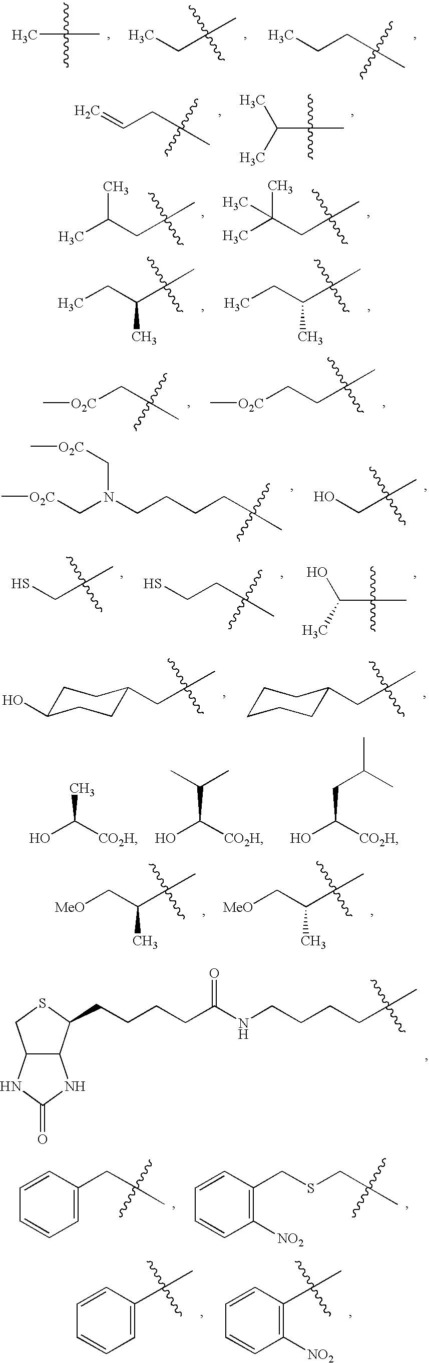

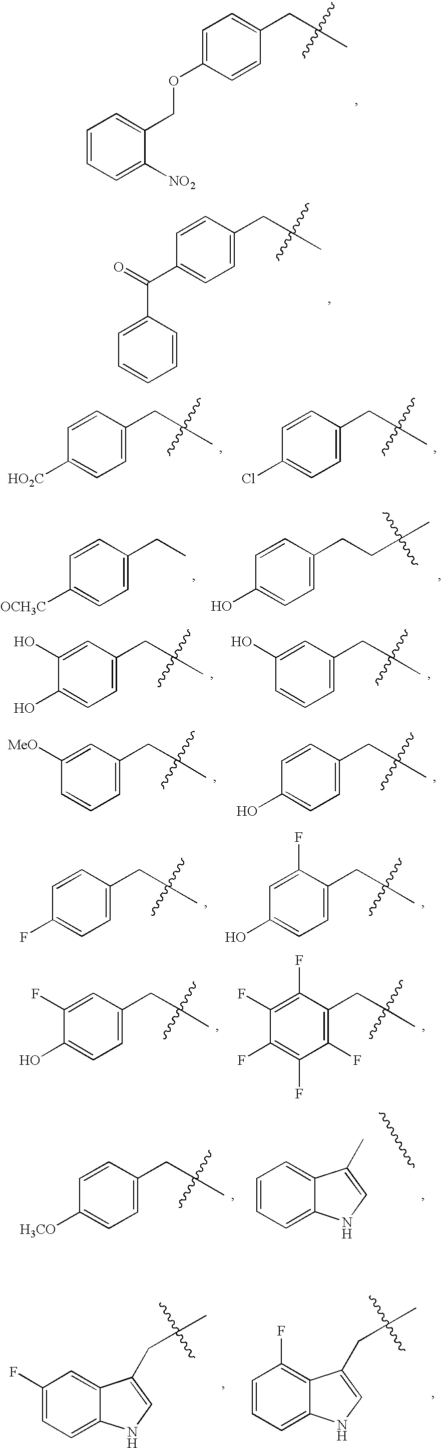

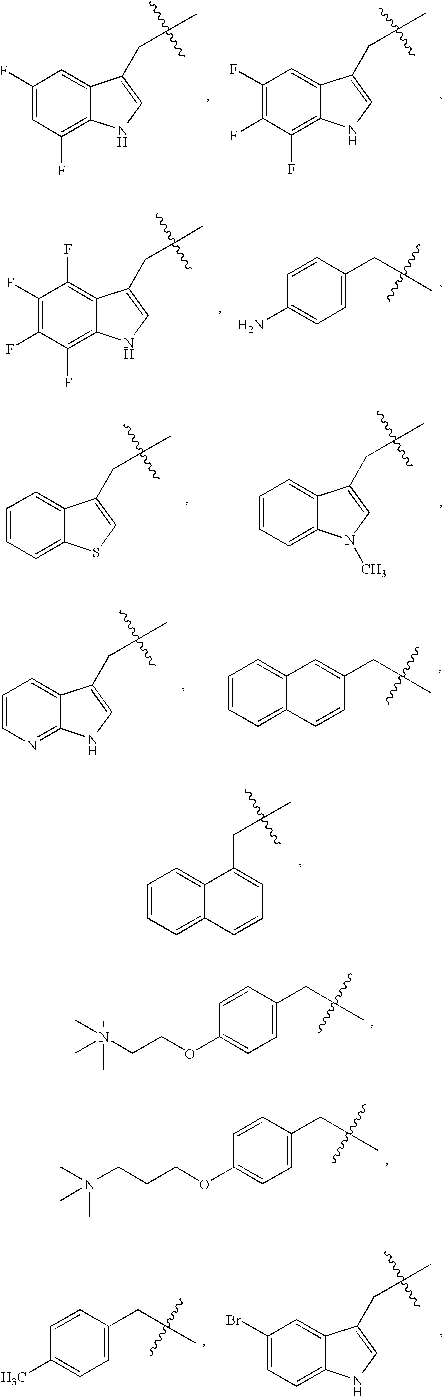

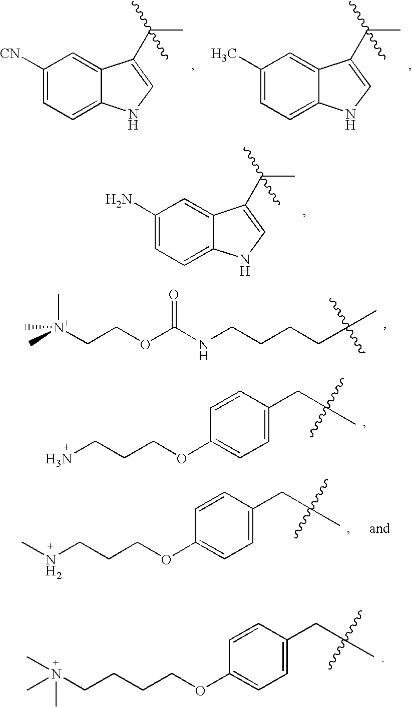

10. The method of claim 7 wherein the unnatural amino acids are selected from the following Formula (II): 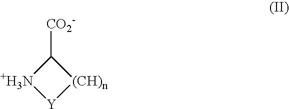
wherein Y is CH₂, (CH)ₙ, N, O, or S, and n is 1 or 2.

11. The method of claim 10 wherein the unnatural amino acid is selected from the group consisting of:

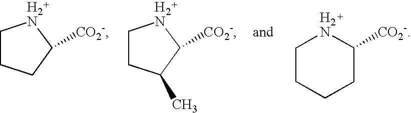

12. A method for determining the impact of an amino acid change on the activity of a native receptor comprising:
a) incorporating an unnatural amino acid into a site of the receptor to form an altered receptor; and
b) comparing the effect of a selected compound upon the altered receptor and upon the native receptor to determine the impact of the amino acid change.

13. The method of claim 12 wherein the unnatural amino acid is incorporated by site-directed mutagenesis or nonsense codon suppression.

14. The method of claim 12 wherein the unnatural amino acid replaces an amino acid in the receptor.

15. The method of claim 12 wherein the unnatural amino acid is inserted into the receptor.

16. The method of claim 12 wherein one or more unnatural amino acids are incorporated into the receptor.

17. The method of claim 12 wherein the unnatural amino acid is incorporated into an allosteric site of the receptor.

18. The method of claim 12 wherein the unnatural amino acid is incorporated into an endogenous binding site of the receptor.

19. The method of claim 12 wherein the effect is the extent of the binding of the selected compound to the altered and native receptors.

20. The method of claim 12 wherein the effect is the extent of the receptor function or compound/receptor interaction of the altered and native receptors.

21. The method of claim 12 wherein the effect is a physiological activity.

22. The method of claim 12 wherein said unnatural amino acid is of Formula (I): 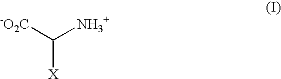
where X is selected from the group consisting of:

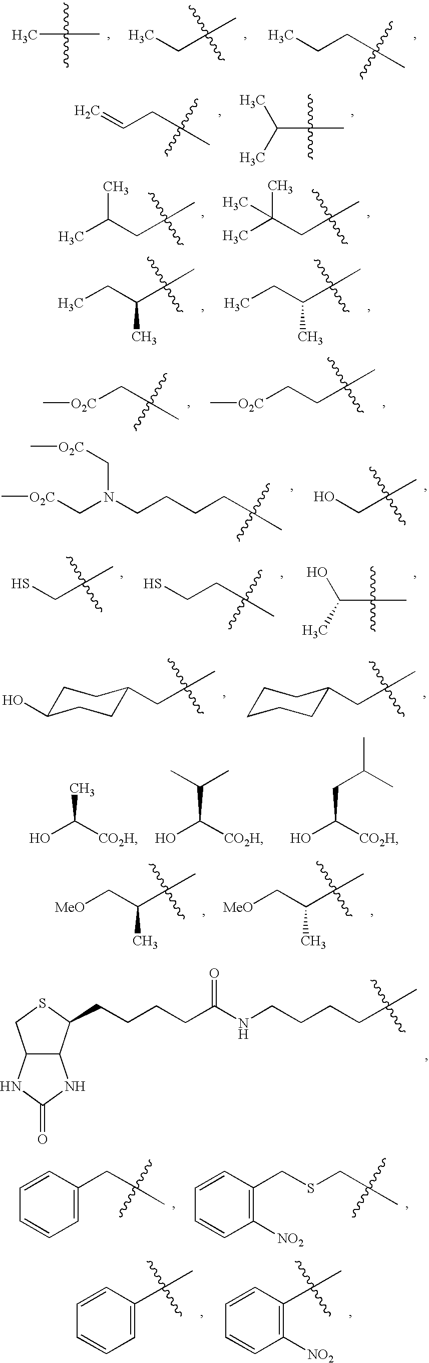

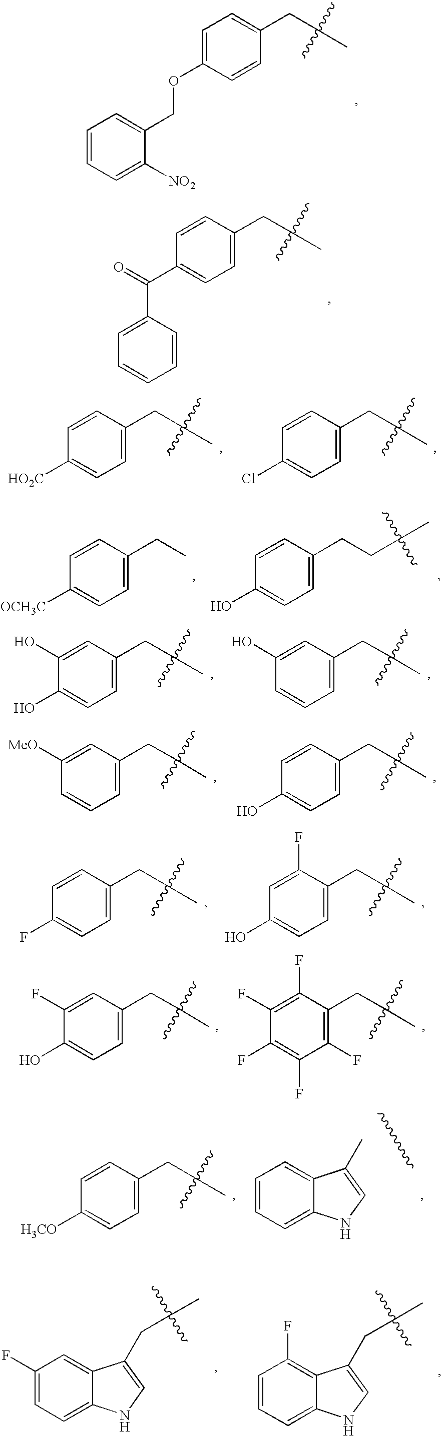

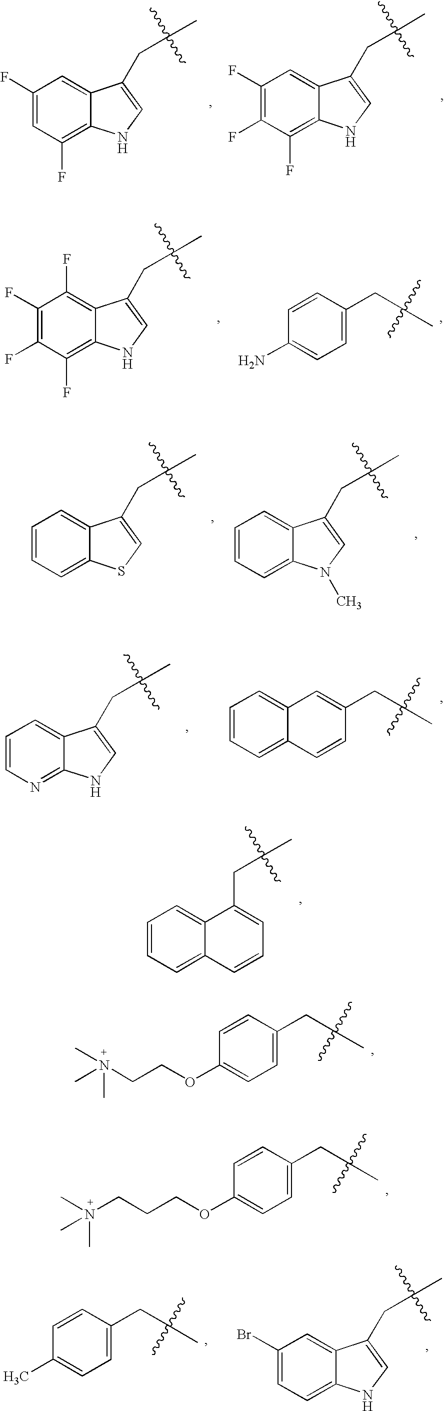

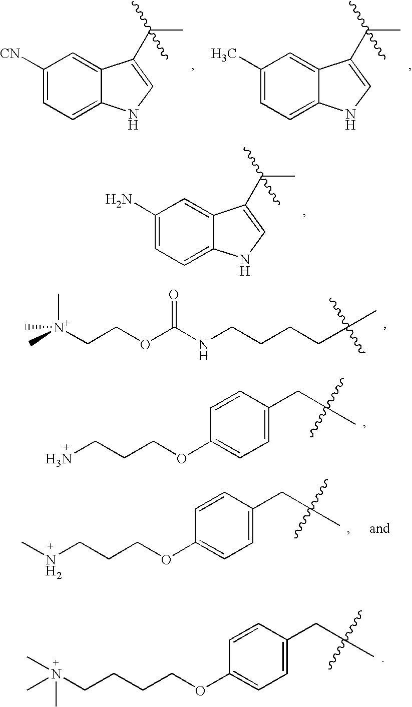

23. The method of claim 12 wherein the unnatural amino acid is of Formula (II): 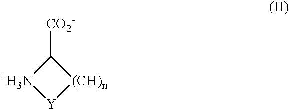
wherein Y is CH₂, (CH)ₙ, N, O, or S, and n is 1 or 2.

24. The method of claim 23 wherein Y is selected from the group consisting of:

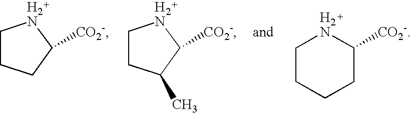

25. The method of claim 12 wherein the receptors are expressed in Xenopus oocytes.

26. The method of claim 12 wherein the receptors are ligand-gated ion channel receptors or G-protein-coupled receptors.


SEQUENCE LISTING (8 sequences)

SEQ ID NO: 1 - DNA, 34 bp, T. thermophila tRNAGln CUA G73
aattcgtaatacgactctactataggttctatag

SEQ ID NO: 2 - DNA, 25 bp, T. thermophila tRNAGln CUA G73
agaacctatagtgagtcgtattacg

SEQ ID NO: 3 - DNA, 28 bp, T. thermophila tRNAGln CUA G73
tatagcggttagtactggggactctaaa

SEQ ID NO: 4 - DNA, 28 bp, T. thermophila tRNAGln CUA G73
gagtccccagtactaaccgctatactat

SEQ ID NO: 5 - DNA, 18 bp, T. thermophila tRNAGln CUA G73
tcccttgacctgggttcg

SEQ ID NO: 6 - DNA, 18 bp, T. thermophila tRNAGln CUA G73
cccaggtcaagggattta

SEQ ID NO: 7 - DNA, 32 bp, T. thermophila tRNAGln CUA G73
aatcccagtaggaccgccatgagacccatccg

SEQ ID NO: 8 - DNA, 39 bp, T. thermophila tRNAGln CUA G73
gatccggatgggtctcatggcggtcctactgggattcga